(19) United States
(12) Patent Application Publication
(10) Pub. No.: US 2022/0334129 A1
(43) Pub. Date: Oct. 20, 2022

(54) Method for identifying T-cell epitopes

(71) Applicant: Evaxion Biotech A/S, Hørsholm (DK)

(72) Inventors: Jens Kringelum, Hørsholm (DK); Emma Jappe, Hørsholm (DK); Christian Garde, Hørsholm (DK); Anthony Purcell, Viewbank (AU); Sri Ramarathinam, Parkville (AU); Nathan Croft, South Yarra, Melbourne (AU)

(21) Appl. No.: 17/642,335

(22) PCT Filed: Sep. 11, 2020
(86) PCT No.: PCT/EP2020/075539

§ 371 Date: Mar. 11, 2022

(30) Foreign Application Priority Data
Sep. 13, 2019 (EP) 19197306.4
Jul. 14, 2020 (EP) 20185772.9

(51) Int. Cl. G01N 33/68; G16B 40/10; G16B 15/30

(52) U.S. Cl. CPC G01N 33/6878; G01N 33/6848; G16B 40/10; G16B 15/30

(57) ABSTRACT

A method for T-cell epitope prediction where quantitative scores of stability in the binding between peptides and MHC molecules are integrated into the derivation of the likelihood that a peptide of defined amino acid sequence constitutes a T-cell epitope. Preferably, stability data are obtained an MS-based method for identification of MHC binding peptides, where the binding capability is quantitatively assessed to allow distinction between stably binding peptides and peptides that are unlikely to be presented to T-cells. The method includes a step of time-course or thermostability testing of naturally processed peptides bound to MHC. Also disclosed are methods for preparation of personalized immunogenic compositions, methods of therapeutic treatment of malignancies, and a computer system that implements the T-cell epitope prediction method.

### FIELD OF THE INVENTION

The present invention relates to the field of immunology, in particular to the identification of MHC binding peptides that are potential T-cell epitopes.

### BACKGROUND OF THE INVENTION

Treatment of malignant neoplasms in patients has traditionally focussed on eradication/removal of the malignant tissue via surgery, radiotherapy, and/or chemotherapy using cytotoxic drugs in dosage regimens that aim at preferential killing of malignant cells over killing of non-malignant cells.

In addition to the use of cytotoxic drugs, more recent approaches have focussed on targeting of specific biologic markers in the cancer cells in order to reduce systemic adverse effects exerted by classical chemotherapy. Monoclonal antibody therapy targeting cancer associated antigens has proven quite effective in prolonging life expectance in a number of malignancies. While being successful drugs, monoclonal antibodies that target cancer associated antigens or antigen can by their nature only be developed to target expression products that are known and appear in a plurality of patients, meaning that the vast majority of cancer specific antigens cannot be addressed by this type of therapy, because a large number of cancer specific antigens only appear in tumours from one single patient, cf. below.

As early as in the late 1950'ies the theory of immunosurveillance proposed by Burnet and Thomas suggested that lymphocytes recognizes and eliminates autologous cells—including cancer cells—that exhibit altered antigenic determinants, and it is today generally accepted that the immune system inhibits carcinogenesis to a high degree. Nevertheless, immunosurveillance is not 100% effective and it is a continuing task to device cancer therapies where the immune system's ability to eradicate cancer cells is sought improved/stimulated.

One approach has been to induce immunity against cancer-associated antigens, but even though this approach has the potential of being promising, it suffers the same drawback as antibody therapy that only a limited number of antigens can be addressed.

Many if not all tumours express mutations. These mutations potentially create new targetable antigens (neo-antigens), which are potentially useful in specific T cell immunotherapy if it is possible to identify the neo-antigens and their antigenic determinants within a clinically relevant time frame. Since it with current technology is possible to fully sequence the genome of cells and to analyse for existence of altered or new expression products, it is possible to design personalized vaccines based on neo-antigens. However, attempts at providing satisfactory clinical endpoints have previously failed.

A key component of effective immunotherapy involves T cell recognition of peptides bound to cell surface major histocompatibility complex (MHC) (Yewdell, Reits and Neefjes, 2003). Peptide immunogenicity is multifaceted, yet current algorithms incorporate only a limited number of features such as peptide-MHC (“pMHC” or “pMHC complex”) binding affinity and antigen processing, offering poor predictive outcome (Mei et al., 2019), (Koşaloğlu-Yalçin et al., 2018; Gfeller et al., 2016).

pMHC stability has been shown to be an important feature, which drives T cell responses (Strønen et al., 2016; Rasmussen et al., 2016). The stability of the pMHC complex is hypothesised to play an important role in the induction of an immune response, since more stable complexes can be presented on the cell surface for a prolonged period of time allowing more effective T cell receptor engagement with the pMHC (Tummino and Copeland, 2008). Several studies have indicated a correlation between pMHC stability and peptide immunogenicity (Strønen et al., 2016; Harndahl et al., 2012; Blaha et al., 2019); however, current pMHC stability assays are biased and suffer experimental limitations in scale.

Importantly, prediction algorithms developed based on selected pMHC stability data have not demonstrated impressive results in predicting T cell epitopes when benchmarked against comparable pMHC affinity predictors (Rasmussen et al., 2016; Jørgensen and Buus, 2014).

One example of state-of-the-art prediction algorithm is NetMHCpan-4.0 (www.cbs.dtu.dk/services/NetMHCpan/; Jurtz V et al., J Immunol (2017), ji1700893; DOI: 10.4049/jimmunol.1700893). This method is trained on a combination of classical MS derived ligands and pMHC affinity data.

Another example is NetMHCstabpan-1.0 (www.cbs.dtu.dk/services/NetMHCstabpan/; Rasmussen M et al., Accepted for J of Immunol, June 2016). This method is trained on a dataset of in vitro pMHC stability measurement using an assay where each peptide is synthesized and complexed to the MHC molecule in vitro. No cell processing is involved in this assay and the environment where the pMHC stability is measured is somewhat artificial. The method in general is less accurate than NetMHCpan-4.0.

U.S. Pat. No. 10,055,540 described a method for identification of neo-epitopes using classical MS detected ligands. Other patent application publications using similar technology are WO 2019/104203, WO 2019/075112, WO 2018/195357 (MHC Class II specific), and WO 2017 106638.

MHCflurry:

(www-sciencedirect-com.proxy.findit.dtu.dk/science/article/piVS2405471218302321) is like NetMHCpan trained on MS detected ligand data and pMHC affinities.

A peptide-MHC Class II interaction prediction method is also disclosed in a recent publication Garde C et al., Immunogenetics, DOI: doi.org/10.1007/s00251-019-01122-z. In this publication, naturally processed peptides eluted from MHC Class II are used as part of the training set and assigned the binding target value of 1 if verified as ligands and 0 if negative.

Generally, these prediction systems employ artificial neural networks (ANNs): ANNs can identify non-linear correlations: Quantification of non-linear correlations is not an easy task, since it is difficult to calculate by simple calculation. This is primarily due to non-linear correlations described with more parameters than linear correlations and probably first appear when all features are considered collectively. Hence it is needed to take all features into account in order to catch the dependency across features.

Structure and processing of ANN: FIG. 14 shows a schematic illustration of a generic ANN. Every feature vector delivers its respective feature value to the associated input neuron in the input layer. The input neurons are connected to hidden neurons in the hidden layer and every hidden neuron is connected to the output neuron. Every hidden neuron and output neuron contain a threshold value which, after calculation, together with the associating input and weights, determines the signal to be forwarded. Increased numbers of hidden neurons and numbers of layers of hidden neurons improves the potential to solve more complex problems of an ANN. Layers of ANN can furthermore be combined in non-linear architectures to generate different properties. Examples of such network architectures are Convolutional Neural Networks (CNN) or Long Short-Term Memory (LSTM) networks. Complex networks and multi-layered ANNs are referred to as deep learning algorithms and resulting prediction models utilizing these networks are referred to as deep learning.

Within recent years, the field of mass spectrometry (MS) and the application of MS to the identification of peptides bound to MHC molecules (the immunopeptidome) has undergone impressive development allowing detection of thousands of peptides in one MS run (cf. the detailed protocol presented in Purcell, Ramarathinam and Ternette, 2019). MS allows the study of peptides, which have been processed by the antigen processing machinery within cells and subsequently bound to an MHC molecule expressed on the cell surface; in other words, the peptides identified as MHC binders by this type of technology are the true products of antigen processing. In contrast, older methods used to identify MHC binding peptides often failed to identify naturally processed forms of these peptides. However, despite the advantages of using MS to study the immunopeptidome, many MS-based peptide identification assays typically detect MHC-bound peptides qualitatively, i.e. either the peptide is detected or it is not detected, and hence the current MS-based methods do not provide further information about the suitability of the peptide as an immunogen. Moreover, those methods that are in fact able to provide quantitative data on MHC bound peptides are not able to provide any further indications of the peptides' suitability as immunogens either.

### Object of the Invention

It is an object of embodiments of the invention to provide methods and means for improved identification and prediction of T-cell epitopes.

### SUMMARY OF THE INVENTION

As detailed above, existing MS methods for identification of MHC (in humans termed HLA) binding peptides typically provide qualitative, but not any quantitative information about the binding properties of the identified peptides. In particular, the stability of the complex between the MHC molecule and the peptide is not determined. This is partly due to the fact that the methodology for preparing the peptides for MS detection in essence provides a “snapshot” of the repertoire of MHC-peptide complexes on the surfaces of the cells presenting the peptides (see FIG. 4 in Purcell, Ramarathinam and Ternette, 2019). In addition, Croft et al. (PLoS Pathogens 2014, Wu et al 2019) have shown that peptide abundance is not directly correlated to immunogenicity. Moreover, quantitative measurements of specific pMHC only indirectly provide an indication of stability as they are a product of the levels of the peptide precursor/antigen turnover and the affinity of the peptide for a given MHC. For instance, a relatively unstable pMHC could be abundant if there is a high level supply of the precursor to drive pMHC complex formation. Equally abundant pMHC complexes may accumulate to high levels even with modest precursor supply if the complexes are stable. These two scenarios cannot be distinguished by prior art simple qualitative or quantitative MS-methods.

The present inventors have hence concluded that the MHC-bound peptides identified from a “snapshot” could include peptides that exhibit individual stabilities for their binding to the MHC molecules, and that this could subsequently be reflected in the probabilities of the MHC-peptide complexes being presented effectively to a T-cell. The reasoning is that when a peptide disassociates from the MHC molecule, the chance that the same peptide will subsequently associate with the same or a different MHC molecule is very close to zero (in particular for MHC class I binding peptides), in particular under the experimental conditions for isolated pMHC, because the MHC molecules, being heterodimers, require a peptide bound in the peptide binding groove in order to constitute stable complexes. So not only can the bound peptide disassociate from the MHC molecule, but this disassociation has the consequence that the MHC heterodimer will disassociate (into the individual α and β chains in the case of MHC Class II and into the α chain and β2-microglobulin in the case of MHC Class I). Therefore, if the snapshot of the peptides presented by a cell's MHC repertoire could include peptides that exhibit low stability for MHC binding at physiological conditions (with the consequence that these peptides are not stably present on the cell) such peptides would therefore stand small chances of being effective T-cell epitopes. Also, it is considered likely by the present inventors that some of the identified peptides would conversely exhibit a high degree of stability for their binding the MHC molecule, which could be reflected in an increased chance that such peptides would be ultimately presented to T-cells. So if ways could be devised that would allow not only a qualitative determination of naturally processed MHC-binding peptides but also a quantitative measure of their stability as MHC binders in the natural context of the cell environment, this would in turn enable a rational selection of peptide sequences, e.g. for the purpose of rational vaccine preparation and design.

Importantly, the present inventors have found that data sets comprising 1) the amino acid sequences of potential T-cell epitopes and 2) a measure each potential epitope's stability for binding to one or more selected MHC molecule(s) adds to be information that is integrated when evaluating the immunogenicity of potential T-cell immunogens and that this significantly improves T-cell epitope prediction. This was found after investigating whether inclusion of data, which are obtained from experiments carried out with a modified experimental protocol that is conceptually based on the one set forth in Purcell, Ramarathinam and Ternette, 2019, could be used to improve existing methods for T-cell epitope identification in silico. Interestingly and as shown in FIG. 5, the experimental method disclosed herein for stability testing is capable of identifying strong binders for MHC molecules that are not identified when using a known T-cell epitope predictor (netMHCpan4.0, available online at www.cbs.dtu.dk/services/NetMHCpan/), and the method disclosed herein for stability testing also demonstrates that certain predicted MHC binding peptides are very poor binders in practice; this underscores that incorporation of pMHC stability data will improve T-cell epitope prediction.

The modified experimental protocol for pHMC stability testing, which is the subject of a co-pending patent application filed simultaneously with the present application, incorporates a “small-scale approach” in order to simultaneously carry out multiple elutions of naturally processed and presented peptides, enabling the investigation of many conditions in one experimental setup rather than simply having a snapshot of the peptides bound to the surface MHC molecules at a given point in time. To this end, the protocol is modified to investigate the number of detectable MHC-bound peptides as a function of time between cell lysis and isolation of MHC-peptide complexes. In another set of experiments, the protocol was modified to investigate the influence of temperature (or other factor contributing to entropy) after cell lysis on the amount of detectable MHC-bound peptides. The protocol is set forth in the present example section as one example of a method which can provide stability scores for a particular binding between a peptide and an MHC molecule.

It has in these experiments with the modified protocol been found that in the context of the immunopeptidome, mass spectrometry analysis (MS) can be used to study the stability of the pMHC. By incubating cell lysates for longer periods of time or at different temperatures (or other entropy modifying conditions), the change in pMHC binding over time or temperature can be studied and directly applied to determine the stability of the individual pMHC complexes. So, rather than carrying out pMHC complex isolation (as described in Purcell 2019) immediately following cell lysis, cell lysates can be incubated for different periods of time or different entropy conditions in order to study the change in pMHC binding, which can be directly applied to determine the stability of the individual pMHC complexes. In turn, this provides a stability score for each investigated peptide, which can then be ranked with respect to their stability for binding to one or more MHC molecules.

The present invention is however not limited to use of data sets from this exact modified protocol—any assay that would be able to provide knowledge about stability of (multiple different) peptides binding to MHC molecules could in practice be the source of data that can be integrated into methods and systems that identify T-cell epitopes based from genome and/or transcriptome data. What has successfully been demonstrated by the present inventors is that T-cell epitope prediction is significantly improved if stability data for defined peptide sequences are incorporated as part of the basis for the identification of T-cell epitopes.

So, in a first aspect the present invention relates to a method for identification of at least one malignant cell-derived peptide, which comprises or consists of a potential T-cell epitope that binds to at least one MHC molecule in an individual, which harbours the malignant cell, the method comprising
a. comparing proteinaceous expression products of said individual's non-malignant cells with proteinaceous expression products of said individual's malignant cells and identifying a set of proteinaceous expression products that are expression products of the malignant cells but not of the non-malignant cells, and
b. identifying the at least one malignant cell-derived peptide as one having 1) an amino acid sequence, which is present in a proteinaceous expression product in the set and not present in any expression product of the non-malignant cells, and 2) a high likelihood of being a natural product of antigen processing and an effective binder of the at least one MHC molecule when compared to the likelihood of other peptides having amino acid sequences present in a proteinaceous expression in the set,

wherein likelihood in step b is determined by including evaluation of the stability of binding between the at least one peptide and the at least one MHC molecule.

A more general version of the first aspect relates to a method for identification of at least one peptide, which comprises or consists of a potential T-cell epitope that binds to at least one MHC molecule in an individual, and which preferably is present in an expression product of a cell or virus, such as an infectious agent, the method comprising a) identifying a set of proteinaceous expression products from the cell or virus, and b) identifying the at least one peptide as one having a high likelihood of being a natural product of antigen processing and an effective binder of the at least one MHC molecule when compared to the likelihood of other peptides having amino acid sequences present in a proteinaceous expression product in the set, wherein likelihood in step ii is determined by including evaluation of the stability of binding between the at least one peptide and the at least one MHC molecule
In a 2^{nd} aspect, the present invention relates to method for preparing a personalized immunogenic composition for an individual, such as a human patient, suffering from a malignant neoplastic disease, the method comprising the sequential steps of extraction of genetic material from malignant cells and from normal cells in the patient, wherein the genetic material is genomic DNA and/or mRNA, identification of RNA sequences or DNA sequences of expressed genes in the genomic DNA from the individual's malignant and non-malignant cells, deducing amino acid sequences of the protein expression products from the RNA/DNA sequences, identification of at least one malignant cell-derived peptide according to the method of any one of the first aspect of the invention, and subsequently
   admixing the at least one malignant cell-derived peptide with a pharmaceutically acceptable carrier, diluent, vehicle, and/or excipient, or
   preparing a polypeptide, which comprises amino acid sequence(s) of the at least one malignant cell-derived peptide and admixing the polypeptide with a pharmaceutically acceptable carrier, diluent, vehicle, and/or excipient, or
admixing a nucleic acid, such as a plasmid, which comprises nucleotide sequence(s) encoding as expressible product(s) the at least one peptide, with a pharmaceutically acceptable carrier, diluent, vehicle, and/or excipient, or
admixing a nucleic acid, such as a plasmid, comprises a nucleotide sequence which encodes as an expressible product a polypeptide comprising the amino acid sequence(s) of the at least one peptide, with a pharmaceutically acceptable carrier, diluent, vehicle, and/or excipient, or
   admixing a microorganism or virus, preferably attenuated and/or non-pathogenic, which is capable of expressing nucleotide sequences encoding the amino acid sequences of the at least one malignant cell-derived peptide, with a pharmaceutically acceptable carrier, diluent, vehicle, and/or excipient, or
   admixing a microorganism of virus, preferably attenuated and/or non-pathogenic, which is capable of expressing a nucleotide sequence encoding a polypeptide comprising the amino acid sequences of the at least one malignant cell-derived peptide, with a pharmaceutically acceptable carrier, diluent, vehicle, and/or excipient.

The second aspect also more generally relates to a method for preparing an immunogenic composition, e.g. for therapeutic or prophylactic treatment of a disease caused by an infectious agent, the method comprising identification of at least one peptide—if relevant derived from such an infectious agent—and subsequently
admixing the at least one peptide with a pharmaceutically acceptable carrier, diluent, vehicle, and/or excipient, or
preparing a polypeptide, which comprises amino acid sequence(s) of the at least one peptide and admixing the polypeptide with a pharmaceutically acceptable carrier, diluent, vehicle, and/or excipient, or
admixing a nucleic acid, such as a plasmid, which comprises nucleotide sequence(s) encoding as expressible product(s) the at least one peptide, with a pharmaceutically acceptable carrier, diluent, vehicle, and/or excipient, or
admixing a nucleic acid, such as a plasmid, comprises a nucleotide sequence which encodes as an expressible product a polypeptide comprising the amino acid sequence(s) of the at least one peptide, with a pharmaceutically acceptable carrier, diluent, vehicle, and/or excipient, or
admixing a microorganism or virus, preferably attenuated and/or non-pathogenic, which is capable of expressing nucleotide sequences encoding the amino acid sequence(s) of the at least one peptide, with a pharmaceutically acceptable carrier, diluent, vehicle, and/or excipient, or
admixing a microorganism of virus, preferably attenuated and/or non-pathogenic, which is capable of expressing a nucleotide sequence encoding a polypeptide comprising the amino acid sequences of the at least one malignant cell-derived peptide, with a pharmaceutically acceptable carrier, diluent, vehicle, and/or excipient.

In a 3^{rd} aspect, the present invention relates to a method for therapeutically treating an individual, such as a human patient, suffering from a malignant neoplasm, the method comprising administering an effective amount of a personalized immunogenic composition prepared according the 2^{nd} aspect of the invention to the individual. Likewise, the 3^{rd} aspect also relates to a method for immunizing (e.g. therapeutically or prophylactically) an individual such as a human patient, the method comprising administering an effective amount of a personalized immunogenic composition prepared according to the more general version of the 2^{nd} aspect of the invention.

In a 4^{th} aspect, the present invention relates to computer or computer system comprising
a) an interface for inputting amino acid sequences data and/or nucleotide sequences,
b) if the interface allows input of nucleotide sequences, executable code for identifying coding sequences in nucleotide sequences and generating encoded amino acid sequences therefrom,
c) a storage segment for storing amino acid sequences provided via input from the interface in a and/or the executable code in b) or for storing unique identifiers of the amino acid sequences,
d) executable code, which generates amino acid sequences of peptides, the amino acid sequences of which are extracted from the storage segment in c or from source(s) identified by the unique identifiers,
e) executable code for an artificial neural network, which
   i. evaluates amino acid sequences of potential T-cell epitopes on the basis of a training set comprising a plurality of amino acid sequences of peptides that are presented by at least one MHC molecule as natural products of antigen processing of protein, and for each of the plurality of amino acid sequences of peptides, a score for the stability of binding between the peptide and the at least one MHC molecule, and
   ii. assigns a score of likelihood that an amino acid sequence generated by the executable code in d) is an amino acid sequence of a peptide which is a natural product of antigen processing and a strong binder of the at least one MHC molecule, and

a storage segment for storing and/or an interface for output of the scores of likelihood generated by the artificial neural network in e), so as to enable comparison between the amino acid sequences generated by the executable code in d) with respect to their scores of likelihood.

In a 5^{th} aspect, the present invention relates to computer-readable, preferably non-transitory, medium storing computer-executable code for identifying potential T-cell epitopes, wherein the code is executable by a computer processor to identify RNA sequences or DNA sequences of expressed genes in genomic DNA from malignant and non-malignant cells, deducing amino acid sequences of the protein expression products from the RNA/DNA sequences, comparing proteinaceous expression products non-malignant cells with proteinaceous expression products of malignant cells and identifying a set of proteinaceous expression products that are expression products of the malignant cells but not of the non-malignant cells, and identifying the at least one malignant cell-derived peptide as one having 1) an amino acid sequence, which is present in a proteinaceous expression product in the set and not present in any expression product of the non-malignant cells, and 2) a high likelihood of being a natural product of antigen processing and an effective binder of the at least one MHC molecule when compared to the likelihood of other peptides having amino acid sequences present in a proteinaceous expression in the set,

wherein likelihood in step b) is determined by including evaluation of the stability of binding between the at least one peptide and the at least one MHC molecule.

### LEGENDS TO THE FIGURE

FIG. 1: Schematic overview of the experimental protocol for determining stability of pMHC.

FIG. 2: Example of peptide filtering in Skyline software (example peptide TLTHVIHNL). A spectral library generated in PEAKS® software using 1% FDR provided 1696 peptides (8mers-11mers) was loaded into the Skyline software, and thereafter peptides were filtered and manually picked to ensure correct precursors and transitions.

FIG. 3: Thermal stability curves for naturally processed peptides eluted from complexes between peptides and MHC molecules isolated from the cell line C1R-A*02:01.

X axis is incubation temperature (° C.), Y axis is relative amounts of isolated peptide.

A: Curves for 12 A*02:01 binding peptides with measured Tₘ values (° C.) ranging between 44.90 and 61.40.

B: Curves for 12 B*07:02 binding peptides with Tₘ values (° C.) ranging between 51.78 and 61.99.

C: Curves for 12 peptides binding both A*02:01 (circles) and B*07:02 (triangles) with Tₘ values (° C.) for binding A*02:01 ranging between 45.71 and 58.96 and with Tₘ values (° C.) for binding B*07:02 ranging between 46.85 and 59.81.

FIG. 4: Graphs showing the distribution of normalized Tₘ values for 491 peptides when compared to prior art determination of ligand binding via MS.

FIG. 5: Graph showing comparison of Tₘ values determined according to the present examples and ligand rank score determined with netMHCpan4.0.

a) results for HLA-A*02:01 ligands

b) results for HLA-B*07:02 ligands

FIG. 6: Graph of peak area ratio relative to global standard in Skyline for peptide ALNELLQHV. Bar represents the peak area ratio of the peptides obtained after incubation of cell lysates at 37° C. for 0, 0.5, 1, 1.5, 2, 3, 5 and 24 hours, respectively.

FIG. 7: Peak curves for peptide ALNELLQHV from 8 samples.

Peaks are shown from samples obtained after incubation of cell lysates at 37° C. for 0, 0.5, 1, 1.5, 2, 3, 5 and 24 hours, respectively.

FIG. 8: Decay curves for 6 peptides subjected to incubation at 37° C. for 0, 0.5, 1, 1.5, 2, 3, 5 and 24 hours, respectively.

Curves shown for peptides RLFDEPQLA, SLLESVQKL, FLFQEPRSI, ILLPEPSIRSV, TLITDGMRSV, and FLDENVHFF.

FIG. 9: Correlation between thermal melting point and half-life.

FIG. 10: Precision-Recall curves for 154 confirmed viral HLA-A0201 restricted T-cell epitopes by two neural networks.

Comparison between two models trained with 491 positive ligands for MHC A*02:01 and 5000 randomly selected negative peptides. The model architecture was random partitioning, 5-fold CV(nnalign), 10, 20, 30, 40 50, and 60 hidden neurons (a consensus model). Blue curve (ligand) shows the precision vs. recall of a model trained with qualitative binding data (binding/no binding), the red curve shows the precision vs. recall of a model trained with stability data using Tₘ as stability score.

Evaluation data was 154 positive T cell epitopes and 770 negatives.

FIG. 11: Precision-Recall curves for 154 confirmed viral HLA-A0201 restricted T-cell epitopes by two neural networks.

Comparison between two models both trained with 11,717 filtered MS ligands (from a public dataset) and 60,000 negatives (randomly sampled), 50 training epochs, burn-in, and thereafter with 491 positive ligands for MHC A*02:01 and 5000 randomly selected negative peptides. Blue curve (ligand) shows the precision vs. recall of a model trained with qualitative binding data (binding/no binding), the red curve shows the precision vs. recall of a model trained with stability data.

Evaluation data was 154 positive T cell epitopes and 770 negatives.

FIG. 12: Precision-Recall curves for 42 confirmed HLA-A0201 restricted T-cell neo-epitopes by two neural networks.

Comparison between two models trained with 491 positive ligands for MHC A*02:01 and 5000 randomly selected negative peptides. The model architecture: random partitioning, 5-fold CV(nnalign), 10, 20, 30, 40 50, and 60 hidden neurons (a consensus model). Blue curve (ligand) shows the precision vs. recall of a model trained with qualitative binding data, the red curve shows the precision vs. recall of a model trained with stability data.

Evaluation data was 42 positive neoepitopes (HLA-A0201 restricted, curated from the literature), 370 negatives (randomly sampled from cancer T cell epitope source proteins from IEDB).

FIG. 13: Precision-Recall curves for 154 confirmed viral HLA-A0201 restricted T-cell epitopes by two neural networks.

Comparison between two models both trained with 11,717 filtered MS ligands (from a public dataset) and 60,000 negatives (randomly sampled), 50 training epochs, burn-in, and thereafter with 491 positive ligands for MHC A*02:01 and 5000 randomly selected negative peptides. Blue curve (ligand) shows the precision vs. recall of a model trained with qualitative binding data (binding/no binding), the red curve shows the precision vs. recall of a model trained with stability data.

Evaluation data was 42 positive neoepitopes (HLA-A0201 restricted, curated from the literature), 370 negatives (randomly sampled from cancer T cell epitope source proteins from IEDB).

FIG. 14: Illustration of a simple neural network with 1 hidden layer of neurons.

Simple representation of a feedforward ANN with four neurons in the input layer, three neurons in the hidden layer and one in the output layer. The signal received from each of the neurons in the previous layer is summed and a bias added. An activation function g(x) is used to pass this information forward in the network.

### DETAILED DISCLOSURE OF THE INVENTION

### Definitions

An “artificial neural network” (ANN) is an executing computer program, which—roughly speaking—is mimicking the architecture of the human brain, in particular of the organization and interaction of neurons in the cerebral cortex. Any ANN contains an input layer that receive data, a number of hidden layers, and an output layer. A processor (“neuron”) in each layer receives input from multiple neurons in other layers in the form of bitwise information (1 or 0) and can only respond by outputting 1 and 0 to other neurons. Each neuron evaluates the sum of input according to a sigmoid evaluation function, which the network is programmed to modify based on “training sets” of data and correct results—if the output layer provides an incorrect result from an input, the evaluation functions are modified throughout the network until the network has been fully trained. A review of the technology can e.g. be found at neuralnetworksanddeeplearning.com/chap1.html. Layers of ANN can be combined in non-linear architectures to generate networks with different properties. Examples of such network architectures are Convolutional Neural Networks (CNN) or Long Short-Term Memory (LSTM) networks. Complex networks and multi-layered ANNs are referred to as deep learning algorithms and resulting prediction models utilizing these networks are referred to as deep learning.

A “peptide” is in the present context a polyamino acid having a length which allows it to fit into the binding groove of an MHC molecule. That is, if the MHC molecule is of class I, the peptides that can bind typically have lengths ranging between 8 and 11 amino acid residues, due to the physical form of the peptide binding cleft. If the MHC molecule is of class II, the peptide has, typically, a minimum length of 9-13 amino acids, but can be considerably longer because the peptide binding cleft in MHC Class II molecules allows for an “overhang”.

An MHC molecule (major histocompatibility molecule) is a tissue antigen expressed by nucleated cells in vertebrates, which binds to peptide antigens and displays (“presents”) the antigens to T-cells carrying T-cell receptors. MHC class I is expressed by all nucleated cells and primarily present proteolytically degraded protein fragments derived from proteins present in the cell. MHC class II is expressed by professional antigen presenting cells that typically take up extracellular protein, degrade it with lysosomal proteases, and present protein fragments on the surface. In humans, the MHC molecules are known as human leukocyte antigens (HLA), which in the present invention are the preferred MHC molecules to evaluate binding to.

T “T-cell epitope” is an MHC binding peptide, which is recognized as foreign (non-self) by a T-cell in a vertebrate due to specific binding between a T-cell receptor and the cell carrying the MHC-peptide complex on its surface. Hence, a peptide, which constitutes a T-cell epitope in one individual will not necessarily be a T-cell epitope in a different individual of the same species. First of all, two individuals having differing MHC molecules that bind different sets of peptides, do not necessarily present the same peptides complexed to MHC, and further, if a peptide is autologous in one of the individuals it may not be able to bind any T-cell receptor.

A “potential T-cell epitope” is a peptide, which exhibits a high likelihood of being recognized as non-self in an individual.

“Naturally processed peptides” are in the present context peptides that can be eluted from an MHC-carrying cell after the peptides have emerged as products of antigen processing by the MHC-carrying cell. Thus, a naturally processed peptide is not simply a peptide, which can form a complex with an MHC molecule. Rather, the naturally processed peptide is by nature a degradation product from the cell's antigen processing machinery. In most prior art methods where peptide-MHC complex formation is measured, peptides—often synthetic—are complexed directly with MHC. This approach can provide for useful insights into peptide-MHC binding, but it does not provide any indication that the MHC binding peptides would or could ever be presented in an MHC context in vivo after processing of a protein antigen (Rock, K. L., Reits, E, and Neefjes J. (2016); Neefjes, J, Jongsma, Paul, P and Bakke, O (2011)).

A “recall” (R)value is the ratio between true positives and the sum of true positives and false negatives, that is R=tp/(tp+fn), used in precision-recall studies. The “precision” (“P) is the ratio between true positives and the sum of true positives and false positives, that is P=tp/(tp+fp).

“AUC” is in the present context the area under the receiver operating characteristic (ROC) curve precision-recall curve, and “AUC0.1” is the area under the ROC curve where the false positive rate (FPR)≤0.1.

An “AP” (average precision) value is defined as Σₙ(Rₙ−Rₙ₋₁)Pₙ.

### SPECIFIC EMBODIMENTS OF THE INVENTION

### Embodiments of the First Aspect of the Invention

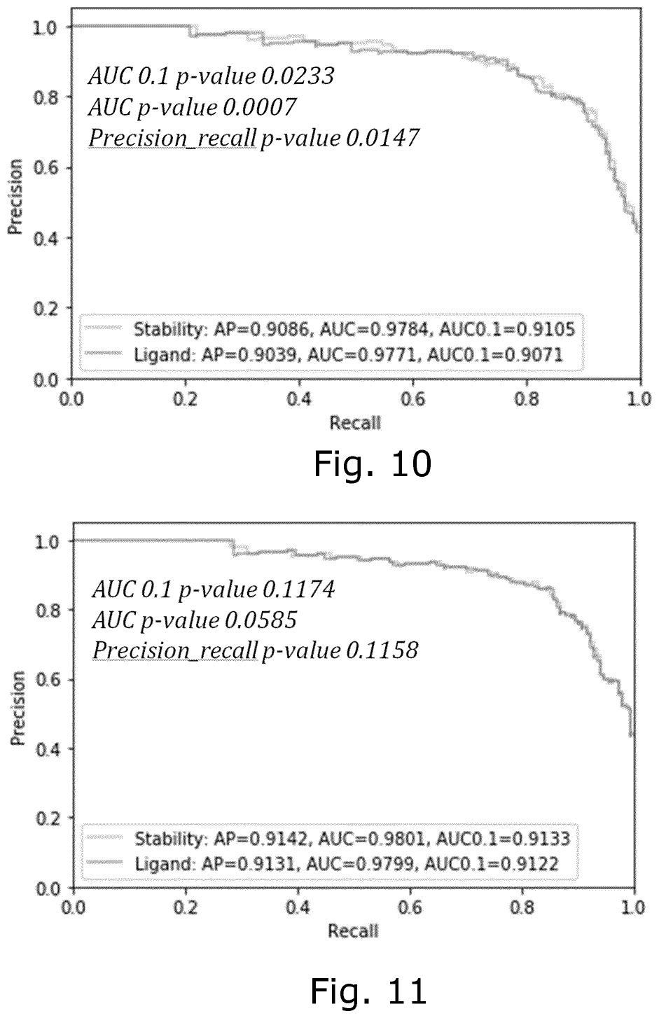

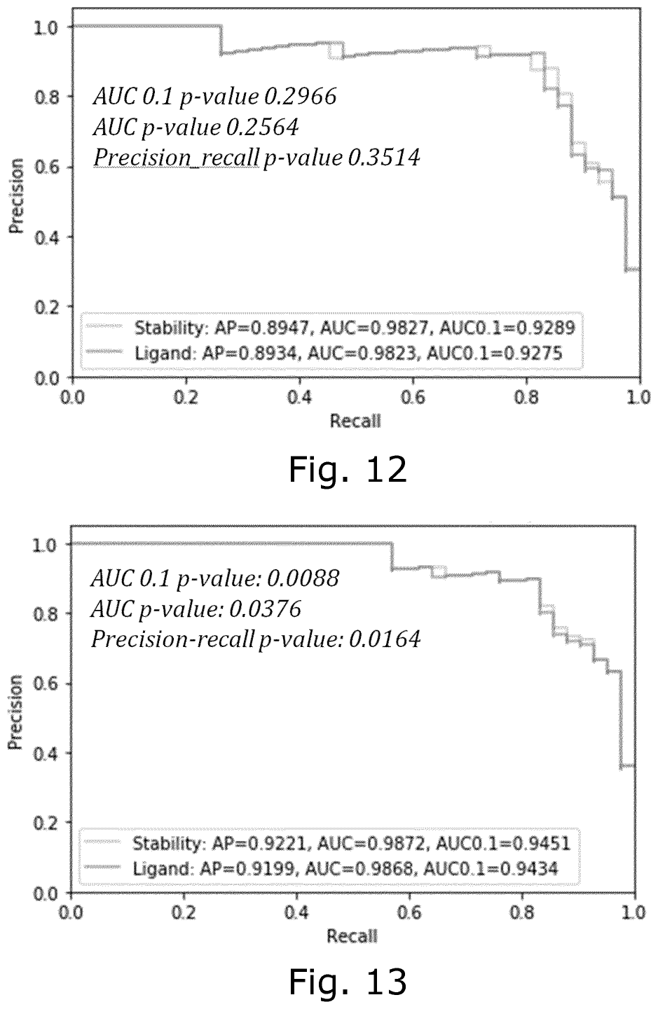

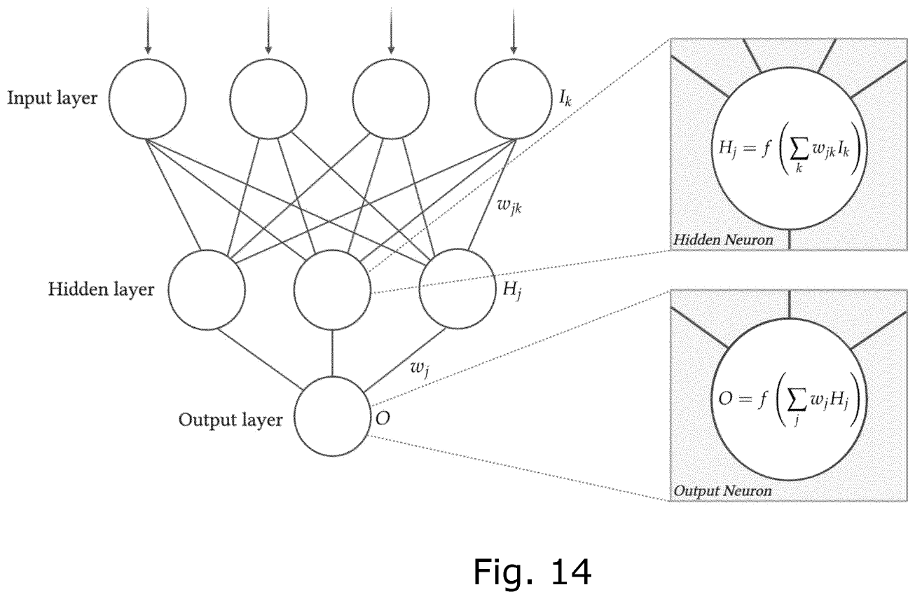

The first aspect of the invention set forth above is based on the finding that incorporation of stability data for pMHC provide for significantly improved precision-recall data when testing neural networks and other computer-implemented algorithms to predict potential T-cell epitopes. As evident from FIGS. 10-13, the AUC values, which provide an indication of the quality of the prediction algorithm, are consistently better for the neural network models that have been trained using stability data.

In principle, step a) can merely be carried out by comparing protein sequences to identify differences between normal and malignant cell protein, but in practice it is often more convenient to identify DNA sequences of expressed genes in the genomic DNA from the individual's malignant and non-malignant cells or to identify mRNA sequences from the individual's malignant and non-malignant cells; this allows deduction of amino acid sequences of the protein expression products. Since it today is possible to rapidly sequence a complete human genome or to obtain mRNA form cells, this approach of using the coding sequences adds to the speed of which the method can be carried out in practice. To deduce the encoded polypeptides' amino acid sequences is a simple matter of applying the genetic code.

As indicated above, a more general version of the first aspect of the invention relates to a method for identification of at least one peptide, which comprises or consists of a potential T-cell epitope that binds to at least one MHC molecule in an individual, and which preferably is present in an expression product of a cell or virus, such as an infectious agent, the method comprising a) identifying a set of proteinaceous expression products from the cell or virus, and b) identifying the at least one peptide as one having a high likelihood of being a natural product of antigen processing and an effective binder of the at least one MHC molecule when compared to the likelihood of other peptides having amino acid sequences present in a proteinaceous expression product in the set, wherein likelihood in step ii is determined by including evaluation of the stability of binding between the at least one peptide and the at least one MHC molecule. This particular version of the first aspect does not necessarily rely on a comparison of amino acid sequences from healthy vs infected or malignant cells, but merely seeks to identify peptides being particularly useful in an immunogenic composition such as a vaccine. Also this aspect includes embodiments wherein step a comprises identification of DNA or RNA sequences of expressed genes in the infectious agent and embodiments wherein step a comprises identifying mRNA sequences encoding proteinaceous expression products and embodiments wherein the amino acid sequences of the protein expression products are deduced from the DNA and/or mRNA sequences. This general version i.a. enables rational design of immunogenic agents that induce T-cell responses, and can hence be useful when designing immunogenic agents such as vaccines that are able to induce immunity against infectious agents, such as bacteria, virus, protozoans (such as amoebae, plasmodia, sporozoans and flagellates), helminths (such as Cestoda, Trematoda, Nematoda), and other parasites.

One preferred way of carrying out the method of the first aspect is to inputting—as part of step b)—the sequences of the proteinaceous expression products into a computer or computer system, which

I) generates amino acid sequences of peptides from the sequences of the proteinaceous expression products by a method comprising 1) subjecting the sequences of the proteinaceous expression products to fragmentation in accordance with the sequence specificity of proteolytic enzymes involved in antigen processing, and/or 2) comparing the sequences of the proteinaceous expression products with known amino acid sequences and the known products of antigen processing thereof, and/or

II) is executing code for an artificial neural network, which identifies amino acid sequences of potential T-cell epitopes on the basis of a training set, which comprises amino acid sequences of known protein antigens and their known T-cell epitopes and the MHC restriction of these.

In general, peptides that are identified in the present invention will be those that are in principle capable of binding MHC molecules. For MHC Class I binders, the peptides will have lengths of 7-13 amino acids (with 8-11 being preferred), whereas MHC Class II binders are peptides that have no defined maximum lengths but minimum lengths ranging from 9-13 amino acids and with maximum lengths of between 15 and 30 amino acid residues. So when using the term peptide throughout the present disclosure, such lengths and functionality is implied.

In both cases I and II—which can be combined and their results consolidated—the output from such an operation is the generation of one or more (normally very large numbers) of amino acid sequences from peptides that are potential binders of MHC molecules. Step b may further comprise generation of a set of likelihoods, where each member of the set of likelihoods indicates the probability that a peptide is a natural product of antigen processing and a strong binder of the at least one MHC molecule. Such a member can both be a single numerical value or a multi-dimensional value (e.g. a vector); in the latter case, the structure of the member can be (p₁, p₂, . . . pₙ), where one of the values (p) is a measure of the probability that the peptide is naturally processed and each of the other values (p) is a probability that the peptide strongly binds a particular MHC molecule—when using the data obtained it is obviously only relevant to include the probabilities for binding to MHC molecules present in the individual in question. Thereby at least one likelihood can be assigned to a plurality of peptides, such as each peptide, for which there has been generated an amino acid sequence from the sequences of the proteinaceous expression products.

The decision on a “high likelihood” in step b) can be expressed relatively or in absolute numbers. Typically a peptide will be considered to have a high likelihood of being a natural product of antigen processing and an effective binder of the at least one MHC molecule when the likelihood is among the top 50% of likelihoods determined, such as among the top 60, 70, 80, and 90%. However, in typical embodiments, a peptide is identified as having high likelihood of being a natural product of antigen processing and an effective binder of the at least one MHC molecule if it is selected from the top 50 likelihoods, such as the top 40, top 30, and the top 25 likelihoods.

The present invention has been tested in neural network models and it is hence preferred that step b) comprises option II discussed above; in that case, it is further preferred that the training set of the neural network comprises 1) a plurality of amino acid sequences of peptides that are presented by at least one MHC molecule as natural products of antigen processing of protein, 2) for each of the plurality of amino acid sequences of peptides, a score for the stability of binding between the peptide and at least one MHC molecule, and, optionally, 3) a plurality of amino acid sequences from irrelevant peptides that are not presented by the at least one MHC molecule. The latter serves as “negative” information in the training set.

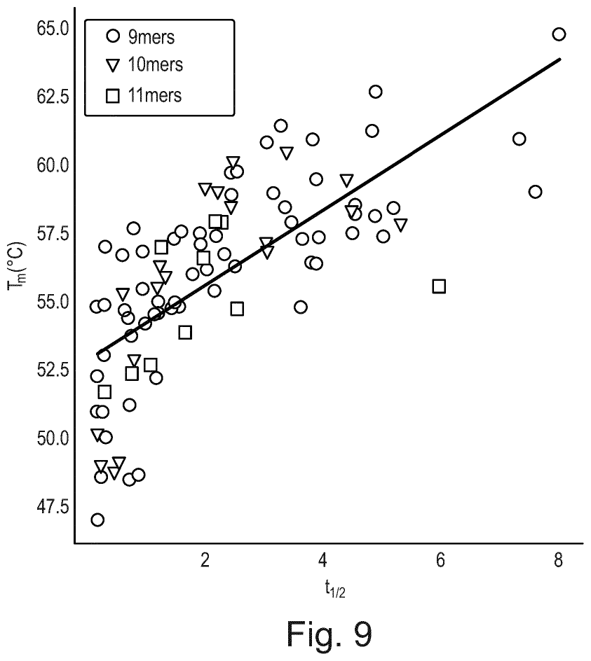

This score for the stability is typically a decay constant for binding between the peptide and the at least one MHC molecule at a selected temperature, or any value being a strictly increasing or decreasing function of the decay constant such as the half-life or the mean lifetime of the peptide binding to the MHC molecule, or Tₘ value for binding between the peptide and the at least one MHC molecule for a selected period of time, or any strictly increasing or decreasing function thereof. As shown in FIG. 9, the two types of values correlate and can hence be used as mutual surrogates. Also, as an alternative to a Tₘ value, it is possible to use a value obtained from a sigmoid curve fitting of other entropy-influencing conditions than temperature.

In some embodiments in line with the laboratory examples set forth herein, the score for stability of binding between the peptide and the at least one MHC molecule is determined by mass spectrometry (MS) analysis of peptides eluted from complexes with MHC molecules, which have been subjected to incubation at defined physicochemical conditions, where incubation time varies between the plurality of samples and where the physicochemical conditions are kept constant between the plurality of samples, or incubation at defined physicochemical conditions, where the incubation time is kept constant between the plurality of samples and where the physicochemical conditions vary between the plurality of samples.

Consequently, the method of the first aspect invention is preferably one where the evaluation of stability of binding between the peptide and the least one MHC molecule is based on a data set defined above, i.e. a data set that integrates a stability score for the binding between multiple pMHCs and MHC molecule(s).

Also in line with the experiments disclosed herein, the data set discussed above is obtained by a method entailing quantitative determination of stability of binding between at least one peptide and an MHC molecule, comprising the subsequent steps of
a) preparing a plurality of samples of cell lysates comprising complexes between MHC molecules and peptides, where the lysates are obtained from a plurality of MHC expressing cells (preferably human cells) that have naturally processed said peptides from protein antigens,
b) subjecting the plurality of samples to the conditions of
   i) incubation at defined physicochemical conditions, where incubation time varies between the plurality of samples and where the physicochemical conditions are kept constant between the plurality of samples, or
   ii) incubation at defined physicochemical conditions, where the incubation time is kept constant between the plurality of samples and where the physicochemical conditions vary between the plurality of samples,
c) isolating complexes between MHC molecules and peptides from the plurality of samples,
d) determining, by mass spectrometric analysis, the at least one peptide's relative quantities in the plurality of samples after step c), and deriving at least one stability score for the at least one peptide based on the quantities determined in step d).

As discussed above, the stability score is typically a decay constant or derivable therefrom or a Tₘ or derivable therefrom. In a separate section below is provided a detailed discussion of this preferred method for generating stability data.

In addition, the score for stability can also be in the form of a probability score indicating the likelihood that the peptide binds stably to the at least one MHC molecule at in vivo physiological conditions. Such a score for stability of binding between the peptide and the at least one MHC molecule is preferably determined by analysis of mass spectrometry (MS) data from peptides eluted from complexes with MHC molecules, wherein the complexes have been subjected to incubation at defined physicochemical conditions for a period of time. As detailed below, such a probability score can be obtained by a simplified MS approach where pMHC is obtained as generally described herein but where only a determination of presence or absence of peptide species is a requirement. For instance, the pMHC can be incubated for a period under conditions that will cause peptides having a relatively low stability for binding to MHC to dissociate from the complex over time. The resulting MS determination of peptides eluted from pMHC will therefore lack information about the dissociated peptides meaning that the peptides that are actually determined to be present are at least more stable. So instead of necessarily quantifying the peptides under a set of different conditions, it is instead possible in a somewhat simper setup to evaluate the presence of peptides.

Therefore, the data set discussed above can also be obtained by a method entailing determination of stability of binding between at least one peptide and an MHC molecule, comprising the subsequent steps of determination of binding between at least one peptide and an MHC molecule by
I) preparing at least one sample of cell lysates comprising complexes between MHC molecules and peptides, where the lysates are obtained from a plurality of MHC expressing cells (preferably human cells) that have naturally processed said peptides from protein antigens, wherein the at least one sample of cell lysates is prepared at a temperature>4° C. and/or wherein the at least one sample of cell lysates is/are incubated for a period of time after obtaining the cell lysates at defined physicochemical conditions at a temperature>0° C., and
   II) determining, by mass spectrometric analysis, whether the at least one peptide is present as part of a complex in the at least one sample after step I).

The at least one MHC molecule is typically an MHC Class I molecule or an MHC Class II molecule, and in both cases preferably an HLA molecule.

### Embodiments of the 2^{nd} Aspect of the Invention

This aspect relates to a method for preparing a personalized immunogenic composition for an individual, such as a human patient, suffering from a malignant neoplastic disease, the method comprising the sequential steps of extraction of genetic material from malignant cells and from normal cells in the patient, wherein the genetic material is genomic DNA and/or mRNA, identification of RNA sequences or DNA sequences of expressed genes in the genomic DNA from the individual's malignant and non-malignant cells, deducing amino acid sequences of the protein expression products from the RNA/DNA sequences, identification of at least one malignant cell-derived peptide according to the method of the first aspect of the invention, and subsequently
admixing the at least one malignant cell-derived peptide with a pharmaceutically acceptable carrier, diluent, vehicle, and/or excipient,
preparing a polypeptide, which comprises amino acid sequence(s) of the at least one malignant cell-derived peptide and admixing the polypeptide with a pharmaceutically acceptable carrier, diluent, vehicle, and/or excipient,
admixing a nucleic acid (DNA or RNA), such as a plasmid, which is capable of expressing nucleotide sequence(s) encoding the at least one malignant cell-derived peptide, with a pharmaceutically acceptable carrier, diluent, vehicle, and/or excipient,
admixing a nucleic acid (DNA or RNA), such as a plasmid, which is capable of expressing a nucleotide sequence encoding a polypeptide comprising the amino acid sequences of the at least one malignant cell-derived peptide, with a pharmaceutically acceptable carrier, diluent, vehicle, and/or excipient,
admixing a microorganism or virus, preferably attenuated and/or non-pathogenic, which is capable of expressing nucleotide sequences encoding the amino acid sequences of the at least one malignant cell-derived peptide, with a pharmaceutically acceptable carrier, diluent, vehicle, and/or excipient, or
admixing a microorganism of virus, preferably attenuated and/or non-pathogenic, which is capable of expressing a nucleotide sequence encoding a polypeptide comprising the amino acid sequences of the at least one malignant cell-derived peptide, with a pharmaceutically acceptable carrier, diluent, vehicle, and/or excipient.

When the first aspect employs the more general approach of the first aspect of the invention, it relates to a method for preparing an immunogenic composition, e.g. for therapeutic or prophylactic treatment of a disease caused by an infectious agent (cf. above), the method comprising identification of at least one peptide—if relevant derived from such an infectious agent—as discussed above under the general version of the 1^{st} aspect of the invention, and subsequently
admixing the at least one peptide with a pharmaceutically acceptable carrier, diluent, vehicle, and/or excipient, or
preparing a polypeptide, which comprises amino acid sequence(s) of the at least one peptide and admixing the polypeptide with a pharmaceutically acceptable carrier, diluent, vehicle, and/or excipient, or
admixing a nucleic acid (DNA or RNA), such as a plasmid, which is capable of expressing nucleotide sequence(s) encoding the at least one peptide, with a pharmaceutically acceptable carrier, diluent, vehicle, and/or excipient, or
admixing a nucleic acid (DNA or RNA), such as a plasmid, which is capable of expressing a nucleotide sequence encoding a polypeptide comprising the amino acid sequence(s) of the at least one peptide, with a pharmaceutically acceptable carrier, diluent, vehicle, and/or excipient, or
admixing a microorganism or virus, preferably attenuated and/or non-pathogenic, which is capable of expressing nucleotide sequences encoding the amino acid sequence(s) of the at least one peptide, with a pharmaceutically acceptable carrier, diluent, vehicle, and/or excipient, or
admixing a microorganism of virus, preferably attenuated and/or non-pathogenic, which is capable of expressing a nucleotide sequence encoding a polypeptide comprising the amino acid sequences of the at least one malignant cell-derived peptide, with a pharmaceutically acceptable carrier, diluent, vehicle, and/or excipient.

In most cases, this method also entails admixing with an immunological adjuvant.

This aspect thus takes advantage of the findings made in the method of the first aspect of the invention, and provides as a product an immunogenic peptide composition (such as a vaccine) “cocktail”, or a multi-epitope protein construct-based immunogenic composition such as a vaccine, which is produced by methods known per se. Also corresponding nucleic acid or live microorganism/virus form are provided in this aspect.

Immunogenic compositions/vaccines prepared according to the invention typically comprise immunising antigen(s), immunogen(s), polypeptide(s), protein(s) or nucleic acid(s), usually in combination with “pharmaceutically acceptable carriers”, which include any carrier that does not itself induce immune responses harmful to the individual receiving the composition. Suitable carriers are typically large, slowly metabolized macromolecules such as proteins, polysaccharides, polylactic acids, polyglycolic acids, polymeric amino acids, amino acid copolymers, lipid aggregates (such as oil droplets or liposomes), and inactive virus particles.

Such carriers are well known to those of ordinary skill in the art. Additionally, these carriers may function as immune stimulating agents (“adjuvants”). Furthermore, the antigen or immunogen may be conjugated to a bacterial toxoid, such as a toxoid from diphtheria, tetanus, cholera, H. pylori, etc. pathogen, cf. the description of immunogenic carriers supra.

Nucleic acid based immunogenic compositions (made from DNA) can be used in DNA vaccination (also termed nucleic acid vaccination or gene vaccination) (cf. e.g. Robinson & Torres (1997) Seminars in Immunol 9: 271-283; Donnelly et al. (1997) Annu Rev Immunol 15: 617-648). Also RNA vaccination is possible. When administering such formats, an effective dose will be from about 0.01 mg/kg to 50 mg/kg or 0.05 mg/kg to about 10 mg/kg of the DNA or RNA constructs in the individual to whom it is administered

For DNA vaccine preparation, the nucleic acid is typically integrated in a vector, such as an expression plasmid. Vectors of the invention may be used in a host cell to produce a polypeptide of the invention that may subsequently be purified for administration to a subject or the vector may be purified for direct administration to a subject for expression of the protein in the subject (as is the case when administering a nucleic acid vaccine).

Suitable expression vectors can contain a variety of “control sequences,” which refer to nucleic acid sequences necessary for the transcription and possibly translation of an operably linked coding sequence in the vaccinated host organism. In addition to control sequences that govern transcription and translation, vectors and expression vectors may contain nucleic acid sequences that serve other functions as well and are described infra.

1. Promoters and Enhancers

A “promoter” is a control sequence. The promoter is typically a region of a nucleic acid sequence at which initiation and rate of transcription are controlled. It may contain genetic elements at which regulatory proteins and molecules may bind such as RNA polymerase and other transcription factors. The phrases “operatively positioned,” “operatively linked,” “under control,” and “under transcriptional control” mean that a promoter is in a correct functional location and/or orientation in relation to a nucleic acid sequence to control transcriptional initiation and expression of that sequence. A promoter may or may not be used in conjunction with an “enhancer,” which refers to a cis-acting regulatory sequence involved in the transcriptional activation of a nucleic acid sequence.

A promoter may be one naturally associated with a gene or sequence, as may be obtained by isolating the 5′ non-coding sequences located upstream of the coding segment or exon. Such a promoter can be referred to as “endogenous.” Similarly, an enhancer may be one naturally associated with a nucleic acid sequence, located either downstream or upstream of that sequence. Alternatively, certain advantages will be gained by positioning the coding nucleic acid segment under the control of a recombinant or heterologous promoter, which refers to a promoter that is not normally associated with a nucleic acid sequence in its natural environment. A recombinant or heterologous enhancer refers also to an enhancer not normally associated with a nucleic acid sequence in its natural state. Such promoters or enhancers may include promoters or enhancers of other genes, and promoters or enhancers isolated from any other prokaryotic, viral, or eukaryotic cell, and promoters or enhancers not “naturally occurring,” i.e., containing different elements of different transcriptional regulatory regions, and/or mutations that alter expression. In addition to producing nucleic acid sequences of promoters and enhancers synthetically, sequences may be produced using recombinant cloning and/or nucleic acid amplification technology, including PCR™, in connection with the compositions disclosed herein (see U.S. Pat. Nos. 4,683,202, 5,928,906, each incorporated herein by reference).

Naturally, it may be important to employ a promoter and/or enhancer that effectively direct(s) the expression of the DNA segment in the vaccinated individual. Those of skill in the art of molecular biology generally know the use of promoters, enhancers, and cell type combinations for protein expression (see Sambrook et al, 2001, incorporated herein by reference). The promoters employed may be constitutive, tissue-specific, or inducible and in certain embodiments may direct high level expression of the introduced DNA segment.

Examples of inducible elements, which are regions of a nucleic acid sequence that can be activated in response to a specific stimulus, include but are not limited to Immunoglobulin Heavy Chain, Immunoglobulin Light Chain, T Cell Receptor, HLA DQα and/or DQβ, β-Interferon, Interleukin-2, Interleukin-2 Receptor, MHC Class II 5, MHC Class II HLA-DRα, β-Actin, Muscle Creatine Kinase (MCK), Prealbumin (Transthyretin), Elastase I, Metallothionein (MTII), Collagenase, Albumin, α-Fetoprotein, γ-Globin, β-Globin, c-fos, c-HA-ras, Insulin, Neural Cell Adhesion Molecule (NCAM), αl-Antitrypain, H2B (TH2B) Histone, Mouse and/or Type I Collagen, Glucose-Regulated Proteins (GRP94 and GRP78), Rat Growth Hormone, Human Serum Amyloid A (SAA), Troponin I (TN I), Platelet-Derived Growth Factor (PDGF), Duchenne Muscular Dystrophy, SV40, Polyoma, Retroviruses, Papilloma Virus, Hepatitis B Virus, Human Immunodeficiency Virus, Cytomegalovirus (CMV) IE, and Gibbon Ape Leukemia Virus.

Inducible Elements include MT II—Phorbol Ester (TFA)/Heavy metals; MMTV (mouse mammary tumour virus)—Glucocorticoids; β-Interferon—poly(rl)x/poly(rc); Adenovirus 5 E2—EIA; Collagenase—Phorbol Ester (TPA); Stromelysin—Phorbol Ester (TPA); SV40—Phorbol Ester (TPA); Murine MX Gene—Interferon, Newcastle Disease Virus; GRP78 Gene—A23187; α-2-Macroglobulin—IL-6; Vimentin—Serum; MHC Class I Gene H-2κb—Interferon; HSP70—E1A/SV40 Large T Antigen; Proliferin—Phorbol Ester/TPA; Tumor Necrosis Factor—PMA; and Thyroid Stimulating Hormonea Gene—Thyroid Hormone.

Also contemplated as useful in the present invention are the dectin-1 and dectin-2 promoters. Additionally any promoter/enhancer combination (as per the Eukaryotic Promoter Data Base EPDB) could also be used to drive expression.

The particular promoter that is employed to control the expression of peptide or protein encoding polynucleotide of the invention is not believed to be critical, so long as it is capable of expressing the polynucleotide in the vaccinated individual. Where a human cell is targeted, it is preferable to position the polynucleotide coding region adjacent to and under the control of a promoter that is capable of being expressed in a human cell. Generally speaking, such a promoter might include either a bacterial, human or viral promoter.

In various embodiments, the human cytomegalovirus (CMV) immediate early gene promoter, the SV40 early promoter, and the Rous sarcoma virus long terminal repeat can be used to obtain high level expression of a related polynucleotide to this invention. The use of other viral or mammalian cellular or bacterial phage promoters, which are well known in the art, to achieve expression of polynucleotides is contemplated as well.

It is contemplated that a desirable promoter for use with the vector is one that is not down-regulated by cytokines or one that is strong enough that even if down-regulated, it produces an effective amount of the protein/polypeptide of the current invention in a subject to elicit an immune response. Non-limiting examples of these are CMV IE and RSV LTR. In other embodiments, a promoter that is up-regulated in the presence of cytokines is employed. The MHC I promoter increases expression in the presence of IFN-γ.

Tissue specific promoters can be used, particularly if expression is in cells in which expression of an antigen is desirable, such as dendritic cells or macrophages. The mammalian MHC I and MHC II promoters are examples of such tissue-specific promoters. 2. Initiation Signals and Internal Ribosome Binding Sites (IRES)

A specific initiation signal also may be required for efficient translation of coding sequences.

These signals include the ATG initiation codon or adjacent sequences. Exogenous translational control signals, including the ATG initiation codon, may need to be provided. One of ordinary skill in the art would readily be capable of determining this and providing the necessary signals. It is well known that the initiation codon must be “in-frame” with the reading frame of the desired coding sequence to ensure translation of the entire insert. The exogenous translational control signals and initiation codons can be either natural or synthetic and may be operable in bacteria or mammalian cells. The efficiency of expression may be enhanced by the inclusion of appropriate transcription enhancer elements.

In certain embodiments of the invention, the use of internal ribosome entry sites (IRES) elements are used to create multigene, or polycistronic, messages. IRES elements are able to bypass the ribosome scanning model of 5′ methylated Cap dependent translation and begin translation at internal sites. IRES elements from two members of the picornavirus family (polio and encephalomyocarditis) have been described, as well an IRES from a mammalian message. IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message (see U.S. Pat. Nos. 5,925,565 and 5,935,819, herein incorporated by reference).

2. Multiple Cloning Sites

Vectors can include a multiple cloning site (MCS), which is a nucleic acid region that contains multiple restriction enzyme sites, any of which can be used in conjunction with standard recombinant technology to digest the vector. (See Carbonelli et al, 1999, Levenson et al, 1998, and Cocea, 1997, incorporated herein by reference). Frequently, a vector is linearized or fragmented using a restriction enzyme that cuts within the MCS to enable exogenous sequences to be ligated to the vector. Techniques involving restriction enzymes and ligation reactions are well known to those of skill in the art of recombinant technology.

3. Splicing Sites

Most transcribed eukaryotic RNA molecules will undergo RNA splicing to remove introns from the primary transcripts. If relevant in the context of vectors of the present invention, vectors containing genomic eukaryotic sequences may require donor and/or acceptor splicing sites to ensure proper processing of the transcript for protein expression. (See Chandler et al, 1997, incorporated herein by reference).

4. Termination Signals

The vectors or constructs of the present invention will generally comprise at least one termination signal. A “termination signal” or “terminator” is comprised of the DNA sequences involved in specific termination of an RNA transcript by an RNA polymerase. Thus, in certain embodiments a termination signal that ends the production of an RNA transcript is contemplated. A terminator may be necessary in vivo to achieve desirable message levels.

The terminator region may also comprise specific DNA sequences that permit site-specific cleavage of the new transcript so as to expose a polyadenylation site. This signals a specialized endogenous polymerase to add a stretch of about 200 A residues (poly A) to the 3′ end of the transcript. RNA molecules modified with this polyA tail appear to more stable and are translated more efficiently in vertebrates. Thus, in other embodiments involving vertebrates such as humans, it is preferred that that terminator comprises a signal for the cleavage of the RNA, and it is more preferred that the terminator signal promotes polyadenylation of the message.

Terminators contemplated for use in the invention include any known terminator of transcription described herein or known to one of ordinary skill in the art, including but not limited to, for example, the bovine growth hormone terminator or viral termination sequences, such as the SV40 terminator. In certain embodiments, the termination signal may be a lack of transcribable or translatable sequence, such as due to a sequence truncation.

5. Polyadenylation Signals

One will typically include a polyadenylation signal to effect proper polyadenylation of the transcript. The nature of the polyadenylation signal is not believed to be crucial to the successful practice of the invention, and/or any such sequence may be employed. Preferred embodiments include the SV40 polyadenylation signal and/or the bovine growth hormone polyadenylation signal, convenient and/or known to function well in various target cells. Polyadenylation may increase the stability of the transcript or may facilitate cytoplasmic transport.

6. Origins of Replication

In order to propagate a vector in a host cell, it may contain one or more origins of replication sites (often termed “on”), which is a specific nucleic acid sequence at which replication is initiated. Alternatively an autonomously replicating sequence (ARS) can be employed if the host cell is yeast.

7. Selectable and Screenable Markers

In certain embodiments of the invention, cells containing a nucleic acid construct may be identified in vitro or in vivo by encoding a screenable or selectable marker in the expression vector. When transcribed and translated, a marker confers an identifiable change to the cell permitting easy identification of cells containing the expression vector. Generally, a selectable marker is one that confers a property that allows for selection. A positive selectable marker is one in which the presence of the marker allows for its selection, while a negative selectable marker is one in which its presence prevents its selection. An example of a positive selectable marker is a drug resistance marker.

Usually the inclusion of a drug selection marker aids in the cloning and identification of transformants, for example, markers that confer resistance to neomycin, puromycin, hygromycin, DHFR, GPT, zeocin or histidinol are useful selectable markers. In addition to markers conferring a phenotype that allows for the discrimination of transformants based on the implementation of conditions, other types of markers including screenable markers such as GFP for colorimetric analysis. Alternatively, screenable enzymes such as herpes simplex virus thymidine kinase (tk) or chloramphenicol acetyltransferase (CAT) may be utilized. One of skill in the art would also know how to employ immunologic markers that can be used in conjunction with FACS analysis. The marker used is not believed to be important, so long as it is capable of being expressed simultaneously with the nucleic acid encoding a protein of the invention. Further examples of selectable and screenable markers are well known to one of skill in the art.

As an alternative, RNA vectors encoding the immunogenic peptide or polypeptide can be used. A review of the most recent advances using this vaccine format is provided in Pardi N et al. 2018, Nat Rev Drug Discov 17(4): 261-279.

With respect to live vaccine or virus based vaccine formats, these are well known in the art and include attenuated and/or non-pathogenic bacteria (such as mycobacteria, such a M. bovis BCG) and virus (such as poxvirus vaccine vectors, including MVA).

When preparing an immunogenic composition according to the present invention—irrespective of the exact immunogen chosen—the following general considerations apply:

The compositions prepared according to the invention typically contain an immunological adjuvant, which is commonly an aluminium based adjuvant or one of the other adjuvants described in the following: Preferred adjuvants to enhance effectiveness of the composition include, but are not limited to: (1) aluminium salts (alum), such as aluminium hydroxide, aluminium phosphate, aluminium sulphate, etc; (2) oil-in-water emulsion formulations (with or without other specific immune stimulating agents such as muramyl peptides (see below) or bacterial cell wall components), such as for example (a) MF59 (WO 90/14837; Chapter 10 in Vaccine design: the subunit and adjuvant approach, eds. Powell & Newman, Plenum Press 1995), containing 5% Squalene, 0.5% Tween 80, and 0.5% Span 85 (optionally containing various amounts of MTP-PE, although not required) formulated into submicron particles using a microfluidizer such as Model 110Y microfluidizer (Microfluidics, Newton, Mass.), (b) SAF, containing 10% Squalane, 0.4% Tween 80, 5% pluronic-blocked polymer L121, and thr-MDP, either microfluidized into a submicron emulsion or vortexed to generate a larger particle size emulsion, and (c) Ribi adjuvant system (RAS), (Ribi Immunochem, Hamilton, Mont.) containing 2% Squalene, 0.2% Tween 80, and one or more bacterial cell wall components from the group consisting of monophosphoryl lipid A (MPL), trehalose dimycolate (TDM), and cell wall skeleton (CWS), preferably MPL+CWS (Detox™); (3) saponin adjuvants such as Stimulon™ (Cambridge Bioscience, Worcester, Mass.) may be used or particles generated therefrom such as ISCOMs (immune stimulating complexes); (4) Complete Freund's Adjuvant (CFA) and Incomplete Freund's Adjuvant (IFA); (5) cytokines, such as interleukins (e.g. IL-1, IL-2, IL-4, IL-5, IL-6, IL-7, IL-12, etc.), interferons (e.g. gamma interferon), macrophage colony stimulating factor (M-CSF), tumour necrosis factor (TNF), etc.; and (6) other substances that act as immune stimulating agents to enhance the effectiveness of the composition.

As mentioned above, muramyl peptides include, but are not limited to, N-acetyl-muramyl-L-threonyl-D-isoglutamine (thr-MDP), N-acetyl-normuramyl-L-alanyl-D-isoglutamine (nor-MDP), N-acetylmuramyl-L-alanyl-D-isoglutaminyl-L-alanine-2″-2′-dipalmitoyl-sn-glycero-3-hydroxyphosphoryloxy)-ethylamine (MTP-PE), etc.

The immunogenic compositions (e.g. the immunising antigen or immunogen or polypeptide or protein or nucleic acid, pharmaceutically acceptable carrier (and/or diluent and/or vehicle), and adjuvant) typically will contain diluents, such as water, saline, glycerol, ethanol, etc.

Additionally, auxiliary substances, such as wetting or emulsifying agents, pH buffering substances, and the like, may be present in such vehicles.

Pharmaceutical compositions can thus contain a pharmaceutically acceptable carrier. The term “pharmaceutically acceptable carrier” refers to a carrier for administration of a therapeutic agent, such as antibodies or a polypeptide, genes, and other therapeutic agents. The term refers to any pharmaceutical carrier that does not itself induce the production of antibodies harmful to the individual receiving the composition, and which may be administered without undue toxicity. Suitable carriers may be large, slowly metabolized macromolecules such as proteins, polysaccharides, polylactic acids, polyglycolic acids, polymeric amino acids, amino acid copolymers, and inactive virus particles. Such carriers are well known to those of ordinary skill in the art.

Pharmaceutically acceptable salts can be used therein, for example, mineral acid salts such as hydrochlorides, hydrobromides, phosphates, sulphates, and the like; and the salts of organic acids such as acetates, propionates, malonates, benzoates, and the like. A thorough discussion of pharmaceutically acceptable excipients is available in Remington's Pharmaceutical Sciences (Mack Pub. Co., N.J. 1991).

Typically, the immunogenic compositions are prepared as injectables, either as liquid solutions or suspensions; solid forms suitable for solution in, or suspension in, liquid vehicles prior to injection may also be prepared. The preparation also may be emulsified or encapsulated in liposomes for enhanced adjuvant effect, as discussed above under pharmaceutically acceptable carriers.

Immunogenic compositions used as vaccines comprise an immunologically effective amount of the relevant immunogen, as well as any other of the above-mentioned components, as needed. By “immunologically effective amount”, it is meant that the administration of that amount to an individual, either in a single dose or as part of a series, is effective for treatment or prevention. This amount varies depending upon the health and physical condition of the individual to be treated, the taxonomic group of individual to be treated (e.g. nonhuman primate, primate, etc.), the capacity of the individual's immune system to synthesize antibodies or generally mount an immune response, the degree of protection desired, the formulation of the vaccine, the treating doctor's assessment of the medical situation, and other relevant factors. It is expected that the amount of immunogen will fall in a relatively broad range that can be determined through routine trials. However, for the purposes of protein vaccination, the amount administered per immunization is typically in the range between 0.5 μg and 500 mg (however, often not higher than 5,000 μg), and very often in the range between 10 and 200 μg.

The immunogenic compositions are conventionally administered parenterally, eg, by injection, either subcutaneously, intramuscularly, or transdermally/transcutaneously (cf. e.g. WO 98/20734). Additional formulations suitable for other modes of administration include oral, pulmonary and nasal formulations, suppositories, and transdermal applications. In the case of nucleic acid vaccination and antibody treatment, also the intravenous or intraarterial routes may be applicable.

Dosage treatment may be a single dose schedule or a multiple dose schedule, for instance in a prime-boost dosage regimen or in a burst regimen. The vaccine may be administered in conjunction with other immunoregulatory agents as may be convenient or desired.

### Embodiments of the 3^{rd} Aspect of the Invention

One important utility of the 1^{st} and 2^{nd} aspects of the invention is as a tool that aids in the design of personalized therapies for cancer patients and in the design of immunogenic agents such as vaccines that target infectious diseases.

When therapeutically treating an individual, such as a human patient, suffering from a malignant neoplasm, an effective amount of a personalized immunogenic composition prepared according to the second aspect can be administered to the individual. As such, samples are initially obtained from the individual and subjected to analyses that can establish the MHC molecule profile of the individual as well as the differences between the proteome in malignant and non-malignant cells. Likewise, the general immunization method of the 3^{rd} aspect that can target infectious disease also rely on the successful identification of strong MHC binding T-cell epitopes. To this end the methods of the 1^{st} and 2^{nd} aspect and all embodiments thereof are used and the individual is ultimately treated with a specifically tailored immunogenic composition prepared according to the 2^{nd} aspect.

The treatment in its own right follows state of the art procedures with respect to administration routes, dosages, formulation of compositions for administration. It is however preferred that such treatment comprises a plurality of administrations, such as in the form of a prime-boost dosage regimen or a burst dosage regimen as is common when administering therapeutic vaccines.

Also, the route of administration is a matter of choice for the clinician, but the immunogenic composition is typically administered parenterally, such as via injection, either subcutaneously, intramuscularly, or transdermally/transcutaneously. Apart from that, all disclosure above concerning dosages, formulations etc., in relation to the 2^{nd} aspect apply mutatis mutandis to the 3^{rd}. aspect.

### Embodiments of the 4^{th} Aspect of the Invention

This embodiment relates to a computer system or a computer, which is adapted to carry out the method of the first aspect of the invention. It thus includes the necessary features that provides a possibility of inputting or feeding amino acid sequence data or nucleotide sequence data to a permanent or temporary storage segment (or separate storage medium), and it also includes the necessary executable code for generating amino acid sequence encoded by any nucleotide sequences that have been inputted and optionally stored. Since the output of the executable code is at least likelihood member discussed above (i.e. a value or a vector indicating the probability that a given peptide is naturally processed and binds a given MHC molecule, it is necessary that either the corresponding peptide's amino acid sequence is stored of at least that a unique identifier (such as a reference number to an external storage or database) for such a peptide is stored to allow subsequent operations be performed on the amino acid sequence. The executable code which generates the amino acid sequences from the storage segment in c, or from a source to which the unique identifier points, will be configured to generate peptides of defined lengths that match the general criteria describe above (the principal ability to bind MHC molecules of a particular Class and type).

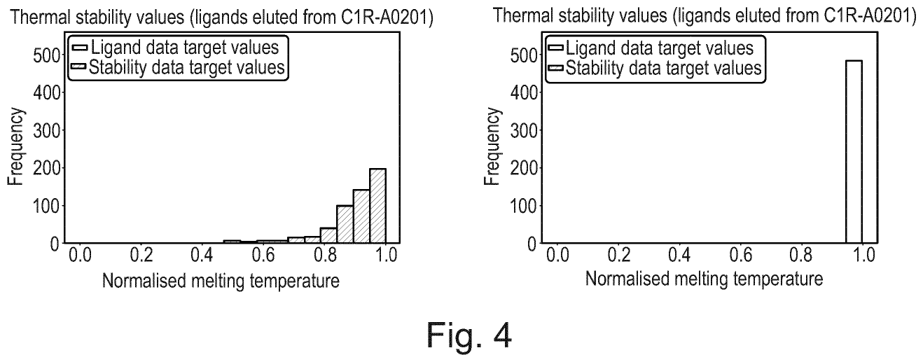

The neural network embedded in the computer/computer system can in essence be any neural network trained to identify MHC ligands—for instance, the presently presented method of the first aspect of the invention could be part of the training set of any of the known methods specifically mentioned in the Background of the Invention section above. In particular, NetMHCpan-4.0 (www.cbs.dtu.dk/services/NetMHCpan/; Jurtz V et al., J Immunol (2017) and the method disclosed in Garde et al. 2019 could both be optimized by including the present training set including stability data. As such, the training set in Garde et al. 2019 would no longer assign affinity values of 0 or 1 for each peptide but instead train with values between 0 and 1—here the transformation used to arrive at FIG. 4 is handy, since it normalizes all Tₘ values to values ranging between 0 and 1.

Finally, the computer system or computer either stores the score of likelihood for each tested amino acid sequence and ensures that the score is tied to the relevant peptide, e.g. by referring to the same unique identifier as would be the case in a typical relation database. Alternatively, output can be presented by providing the amino acid sequence and the likelihood scores relative to one or more MHC molecules.

The interface in a is typically selected from any state-of-the-art input feature, e.g. a manual input device, such as a keyboard, a voice recognition system, a reader of information on a storage medium, a database connection, and a data acquisition system.

In general, the computer system will further comprise the necessary features and elements necessary to carry out the method of the first aspect of the invention, i.e. the code necessary to identify differences between expressed amino acid sequences, identification of natural processing products etc. and executable code that will store the amino acid sequences to be tested.

### Embodiments of the 5^{th} Aspect of the Invention

This aspect relates to a computer executable product, that is, a medium storing executable code for identifying potential T-cell epitopes. As such, the medium stores executable code for carrying out embodiments of the method of the first aspect of the invention.

Therefore it is preferred the executable code I) generates amino acid sequences of peptides from the sequences of the proteinaceous expression products by 1) subjecting the sequences of the proteinaceous expression products to fragmentation in accordance with the sequence specificity of proteolytic enzymes involved in antigen processing, and/or by 2) comparing the sequences of the proteinaceous expression products with known amino acid sequences and known products of antigen processing thereof, and/or II) comprises code for an artificial neural network, which identifies amino acid sequences of potential T-cell epitopes on the basis of a training set, which comprises amino acid sequences of known protein antigens and their known T-cell epitopes.

The executable code thus has features which correspond to the embodiments described above for the 1^{st} aspect of the invention and hence all disclosures relating to steps and features of the 1^{st} aspect applies mutatis mutandis to the executable code of the 5^{th} aspect.

Disclosure Relating to Generation of Data for Stability of pMHC

One suitable method for generating stability data used in the above-described aspects of the invention is a method for quantitative determination of stability of binding between at least one peptide and an MHC molecule, comprising the subsequent steps of
a) preparing a plurality of samples of cell lysates comprising complexes between MHC molecules and peptides, where the lysates are obtained from a plurality of MHC expressing cells (preferably human cells) that have naturally processed said peptides from protein antigens,
b) subjecting the plurality of samples to the conditions of
   i) incubation at defined physicochemical conditions, where incubation time varies between the plurality of samples and where the physicochemical conditions are kept constant between the plurality of samples, or
   ii) incubation at defined physicochemical conditions, where the incubation time is kept constant between the plurality of samples and where the physicochemical conditions vary between the plurality of samples,
c) isolating complexes between MHC molecules and peptides from the plurality of samples,
d) determining, by mass spectrometric analysis, the at least one peptide's relative quantities in the plurality of samples after step c), and
e) deriving at least one stability score for the at least one peptide based on the quantities determined in step d).

This method has proven (cf. the Example section) to provide detailed information about peptides that are natural products of antigen processing in nucleated cells and in particular to provide a means for developers of e.g. peptide-based vaccines and diagnostics to focus on those peptides that are likely to be specifically presented to T-cells by antigen presenting cells for a prolonged period of time, thereby increasing the likelihood of recognition and binding. By subjecting the complexes to step b), it is determined for each complex how its binding properties are under near-physiological conditions over time or under varying entropy conditions, and—importantly—it thereby becomes possible to rationally select peptides for further development based on ranking of their binding properties. This also implies that the at least one peptide normally is a larger number of peptides that each obtain a stability score after being subjected to the stability determination method.

The cells that are initially used to provide the cell lysates in step a) are as a rule pelleted into pellets of 5×10⁷-1×10⁹ cells; however, the number of cells is not crucial, but merely has to be large enough to allow that the subsequent steps provides a sufficiently high number of samples of cell lysates so as to obtain the necessary information in step d). Post lysis of these large pellets, the lysate is divided into the desired number of replicates (each of the same number of cells), which are each subjected to conditions specified in step b). The large pellets can also be used in the protocol described in Purcell et al. 2019 to provide a large spectral library of peptides which serves as reference for the MS analysis carried out in the method for stability determination.

Typically, the MHC-expressing cells are mono-allelic for the MHC molecule; this allows for a definite mapping of peptide binding versus a particular MHC molecule, in humans mapping of peptide binding versus a specific HLA molecule.

When the MHC molecule is an MHC class I molecule, it is preferably selected from HLA-A, HLA-B, and HLA-C. The frequencies of known HLA alleles are provided at www.allelefrequencies.net/hla6006a.asp and since the stability determination method is applicable to any HLA allele, it is e.g. of interest to carry out the stability determination method using the most relevant alleles for the population that is to be vaccinated with peptides.

When the MHC molecule is an MHC class II molecule, it is preferably HLA-DP, HLA-DQ, and HLA-DR.

In general, the stability determination method conceptually follows the general outline of steps for cell preparation/isolation, isolation of complexes, elution of peptides and MS analysis, which is detailed in Purcell et al. 2019. For instance, it is preferred that the plurality of MHC-expressing cells prior to step a) have been isolated/separated from other organic material by centrifugation and optionally have been frozen for storage prior to step a). Freezing the cells should be carried out at sufficiently low temperature to ensure that the cells, and thereby the MHC complexes with peptides, are not degraded—freezing in liquid nitrogen is preferred.

Also, in line with Purcell et al. 2019, step c) preferably comprises isolation of the complexes by means of affinity purification specific for the MHC molecule; detailed protocols are set forth in the examples. I.e. the step utilises a reagent that detects/isolates the intact pMHC complex. This reagent can be an antibody or any molecule that has or mimics the binding properties of an antibody: antibody fragments and variants can be used and also molecular imprinted polymers. Also here it is important that the temperature is kept sufficiently low to ensure integrity of the MHC complexes with the peptides; somewhat unexpectedly, a sufficiently low temperature has proven to be room temperature. In the examples, two different procedures are reported for isolation of the complexes in the large-scale and small-scale experiments, respectively. In the large-scale experiment, the complexes are captured with cross-linked antibodies bound to a matrix in an affinity column and subsequently eluted, thus providing an eluate without capture antibody, whereas the small-scale experiment utilises capture antibody coupled to protein A, where the eluate comprises both the complexes and the antibodies, followed by filtration (to remove antibody). However, in practice the immunoprecipitation method used in the large-scale experiment could be used for the stability determination method, since it is possible to apply it on the lysates that have been subjected to step b). Thus, the exact separation method for isolation of the complexes is not essential.

Steps a) and b) constitute a deviation from/addition to the protocol in Purcell et al. 2019: the preparation of a plurality of samples (typically corresponding to the number of different physiochemical and/or time-course conditions applied in the next step), is novel and necessary in order to investigate the stability of binding between MHC and peptides under a set of different conditions. It is, however, often convenient to utilize the present method in combination with the protocol of Purcell et al. 2019 because this will provide a large spectral peptide library against which the peptides examined in the presently presented method can be analysed.

Since each peptide examined in the later MS step d) cannot be directly quantitatively compared with the other peptides, it is advantageous to investigate the quantity of each peptide relative to its own quantity measured from one of the plurality of samples. In other words the quantities for a peptide determined in step d) are normalized relative to one single of the quantities determined for the peptide—this can e.g. be a median or average value of multiple measured values from peptides subjected to the same circumstances; typically, the quantities are normalized relative to the highest quantity measure for peptide, which for each peptide typically will be the quantity found in the sample subjected to either the shortest incubation time in step b)i) or the quantity determined for the condition that provides the lowest incubation entropy in step b)ii).

When the method of the first aspect in step b) comprises subjecting the plurality of samples to conditions i), it is preferred that the stability score is in the form of a decay constant (λ) for peptide binding to the MHC molecule, or any value being a strictly increasing or decreasing function of the decay constant such as the half-life (t_{1/2}) or the mean lifetime (τ) of the peptide binding to the MHC molecule. As is well-known the decay constant, half-life, and mean life time are related as follows:

$${t}_{1 / 2} = \frac{ln ( 2 )}{λ} = τ ln ( 2 ) .$$

In order to accurately determine a decay constant, the representing MHC-peptide complexes are conveniently fitted to a decay curve (cf. below), with incubation times represented on the X-axis and a quantity measure represented on the Y-axis. It is for practical reasons preferred that data are sampled within 24 hours when incubation of cell lysates is made at body temperature (in the examples incubation times range between 0 hours to 24 hours) but if selecting a different incubation temperature, the incubation times could be longer (if the incubation temperature is lowered) or shorter (if the incubation temperature was increased). Also, some peptides have been observed by the inventors to remain stably bound at physiological conditions even after 24 hours, which is hence not a general limitation. In general, the incubation times can be reduced if the physicochemical constant conditions provide for relative high entropy and vice versa—however, the physicochemical conditions should not be destructive in the sense that they could denature the MHC-peptide complexes.

When the stability determination method comprises subjecting the plurality of samples to conditions ii), it is preferred that the stability score is in the form of a Tₘ value, or any strictly increasing or decreasing function thereof. Use of Tₘ as the stability score presupposes that the physicochemical condition that is varied in step b)ii) is temperature, which is also the preferred embodiment, but the method is not limited to this embodiment. In practice, application of chaotropic agents such as urea, n-butanol, ethanol, guanidinium chloride, lithium perchlorate, lithium acetate, magnesium chloride, phenol, 2-propanol, sodium dodecyl sulphate, and thiourea in various concentrations would also be able to cause the dissociation of the binding between the MHC molecules and their bound peptides, but it would require a somewhat laborious setup (such as hollow fibres that have the complexes stably bound) in order to rapidly interrupt the contact between the complexes and such chaotropic agents so as to ensure that the plurality of samples are subjected to the chaotropic agent for the same period of time. At any rate, the important feature of step b)ii) is to be certain that the MHC-peptide complexes are subjected to conditions that provide different levels of entropy but for defined periods of time.

The duration of the constant incubation time in step b)ii) is not essential as long as it is sufficient to provide a measurable effect of the varying physicochemical conditions on the stability of the complexes. Also, the varied physicochemical condition (such as temperature) must be chosen so as to at least avoid denaturation of the individual polypeptides being part of the MHC complexes thereof—it goes without saying that subjecting pMHC to temperatures or other conditions that would lead to intramolecular destruction (i.e. irreversible denaturation) of protein structure will provide no meaningful results in terms of stability of binding between MHC and peptide. So setting out at—and concentrating on obtaining measurements from—conditions where the stability of pMHC is (almost) exclusively governed by the dissociation of peptides from intact MHC is preferred. In experiments carried out by the inventors (data not shown) it was found that a incubation time in step b)ii) of 5-10 minutes (with about 10 minutes being preferred) provides excellent results. In the present examples, the varied physicochemical condition was temperature, which was varied between body temperature (37° C.) and 73° C., which was effective in providing the necessary information for a melting curve and Tₘ values for individual pMHC complexes.

For both of conditions i) and ii) the choice of physicochemical conditions are preferably made in order to ensure 1) that variations in isolated peptides between conditions can be obtained and 2) that the conditions are not too destructive to provide meaningful results. Hence, the choice of different temperatures are typically made within the interval 1-90° C.—for instance, all incubation temperatures>0° C. detailed below under the description of the “simplified generation of data for stability of pMHC” useful when generating quantitative stability data.

After having carried out step c, the stability determination method can again be carried out essentially as disclosed in Purcell et al. 2019, meaning that it is preferred that step d) comprises tandem mass spectrometric analysis. For this purpose, step c) typically includes a further step of separating peptides from MHC molecules to allow the subsequent MS testing of the isolated peptides. This provides MS data that can subsequently be subjected to further analysis with state of the art software for peptide identification and sequencing (such as the PEAKS® software) and for data independent acquisition quantitative methods (such as the Skyline software (Maclean et al. 2010) and DIA-NN (Demichev et al. 2020)).

An important feature of preferred embodiments of the stability determination method is that step d) comprises that the amino acid sequence of the at least one peptide and a measure of its relative quantity is determined in step d) in each of the plurality of samples. As noted above, this provides the possibility to compare—for each peptide—its relative quantities (using as a reference point its own quantity in one sample or the mean or median of several quantities of the same peptide from samples subjected to identical conditions) in samples that have been subjected to different conditions in step b). When using the expression “relative quantity”, it is meant that the data derived from the stability determination method at least have to provide information about the amount of each peptide subjected to one set of conditions relative to the same peptide subjected to a different set of conditions—this does not exclude that absolute values of quantity may be derived and useful, but in order to derive a stability score, it is not essential to derive an absolute measure of quantity.

The stability score of the at least one peptide is preferably derived by fitting its quantities determined in step d) to a decay curve against time if the plurality of samples have been subjected to conditions i) in step b) or to a sigmoid melting curve against temperature if the plurality of samples have been subjected to conditions ii) in step b).

At least two determinations can be made of stability of binding between at least one peptide and an MHC molecule, wherein one determination comprises subjecting a first plurality of samples to conditions i) in step b) and another determination comprises subjecting a second plurality of samples to conditions ii) in step b). Therefore, at least two stability scores are derived for the at least one peptide in step d), such as a stability scores detailed above. It is however relatively time- and resource-consuming to carry out both types of experiments, and since both sets of conditions will provide the necessary information on the stability between peptides and MHC, it is normally only relevant to carry out one of the two of which the thermostability condition testing has turned out to be the least time-consuming. It is to be noted that the inventors have demonstrated (cf. FIG. 9) that the stability measures obtained from time-course and thermostability studies, respectively, correlate, so that each can be used as a surrogate for the other.

Simplified Generation of Data for Stability of pMHC

As mentioned above, it is also possible to utilise a stability score, which is in the form of a probability score rather than using a determined measure of stability. Such a probability score can be arrived at by (normally qualitative) determination of binding between at least one peptide and an MHC molecule, comprising the subsequent steps of
I) preparing at least one sample of cell lysates comprising complexes between MHC molecules and peptides, where the lysates are obtained from a plurality of MHC expressing cells (preferably human cells) that have naturally processed said peptides from protein antigens, wherein the at least one sample of cell lysates is prepared at a temperature>4° C. and/or wherein the at least one sample of cell lysates is/are incubated for a period of time after obtaining the cell lysates at defined physicochemical conditions at a temperature>0° C.,
II) determining, by mass spectrometric analysis, whether the at least one peptide is present as part of a complex in the at least one sample after step I).

The findings made in relation to the stability determination method disclosed above can be summarized by concluding that naturally processed peptides that are isolated from MHC complexes have different stabilities for binding to the MHC and that those having high stability are more likely to be presented to T-cells by APCs. The simplified data generation method enables exploitation of this finding in a slightly simpler manner than by necessarily determining a stability score derived from multiple measurements of pMHC abundance as described above.

For instance, one set of possible implementations of the simplified data generation method compares the results after step II between samples of peptide-MHC which have been subjected to different levels of entropy, typically different temperature levels, or between samples that have been incubated at physicochemical conditions that allow an appreciable irreversible dissociation of pMHC. Peptides that are not detected beyond a detection threshold at higher entropy levels (or after prolonged incubation) will be considered absent as part of a complex in the sample at these entropy levels or after the incubation period. The end result is ideally that from the original pool of binding peptides present on the MHC expressing cells (which can be considered the reference sample that defines the maximum number of potentially relevant peptides bound to MHC), a fraction thereof will be present as part of a complex in the sample at all entropy levels tested or after even the longest incubation times. These peptides are to be considered “generally stable binders”. It is of note that the highest entropy levels that the complexes are exposed to will not result in denaturation of the MHC structure in the sense that the individual components of the MHC molecule remains largely intact—as mentioned under the discussion data generation method above, an entropy level that will render association between MHC and peptide impossible due to extensive destruction of the intramolecular structure of MHC will not provide any meaningful results. In practice, this means that temperatures exceeding 75° C. should largely be avoided.

An even more simplified version uses only one single determination, preferably at an entropy level close to or higher than the entropy level found at physiological conditions but still at an entropy level that does not result in denaturing of MHC.

Typically, the determination of binding in simplified data generation method is “qualitative” in the sense that only presence or absence of a given peptide is determined. However, it is possible to employ any available quantitative MS determination method, and if such quantitative determination methods are employed, the outcome of the method will be a quantitative determination of the peptide. This emphasizes that the exact choice of MS approach is of limited importance whereas it is essential that the peptides whose presence is determined have been subjected to entropy conditions and/or incubation times that allow for conclusions to be drawn with respect to their stability for binding to MHC molecules.

In the simplified data generation method, the temperature>4° C. is selected from a temperature of about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, about 60, about 61, about 62, about 63, about 64, about 65, about 66, about 67, about 68, about 69, about 70, about 71, about 72, about 73, about 74, about 75, about 76, about 77, about 78, about 79, about 80, about 81, about 82, about 83, about 84 about 85, about 86, about 87, about 88, about 89, and about 90° C. Likewise, the temperature>0° C. is selected from a temperature of about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, about 60, about 61, about 62, about 63, about 64, about 65, about 66, about 67, about 68, about 69, about 70, about 71, about 72, about 73, about 74, about 75, about 76, about 77, about 78, about 79, about 80, about 81, about 82, about 83, about 84 about 85, about 86, about 87, about 88, about 89, and about 90° C.

When incubation for a period of time is employed, the period of time is preferably at least or about 5, about 10, about 20, about 30, about 40, about 50, about 60, about 120, about 240, about 480, about 720, about 960, about 1440, about or 1920, about 2160, about 2880, about 3600, about 4320, about 5040, about 5760, about 6480, about 7200, about 7920, about 8640, about 9360, about 10080, about 10800, or about 11520 minutes. The incubation period is, however, relative to the entropy conditions. If selecting to incubate at relatively low temperatures (<10° C.) or at low entropy levels, incubation times of weeks or even months can be relevant. On the other hand, if selecting to incubate at high entropy levels, very short incubation times can be useful, e.g. as short at 30 seconds, 1 minute, 2 minutes, 3 minutes, and 4 minutes.

In general, the method steps described in detail for the data generation method can where relevant be applied mutatis mutandis to the simplified method, i.e. all details pertaining to the provision and preparation of MHC expressing cells, MHC molecules, complex isolation, peptides isolation and MS procedures are relevant in the simplified method.

In particular, step II preferably comprises the steps of isolating complexes of MHC and peptides, preferably by means of affinity purification specific for the MHC molecule, separating peptides from the complexes and subjecting separated peptides to MS.

A plurality of samples can be prepared wherein lysis conditions and/or incubation conditions favour the preservation of complexes between MHC and peptides to different degrees across the samples. This provides for a number of different MS “fingerprints” of the samples, one for each condition, where peptides are determined to be present or absent in step II. With increasing temperature/entropy levels (or prolonged incubation), a decreasing number of peptides found in step II will be observed—and this allows selection of those peptides that are sufficiently stable by simply selecting those that appear at the higher (preferably all) selected entropy conditions. While this approach does not necessarily provide any indication of the abundance of the stable peptides, it nevertheless provides a simple method for screening of peptides that are not stable. It is understood, however, that the exact choice of MS determination method will dictate whether an indication of abundance can be arrived at or not.

Hence, in in a very simple implementation, the at least one sample is subjected to one single set of lysis and incubation conditions; this set of conditions is preferably one that reflects physiologic conditions in the sense that the physicochemical conditions and the incubation time will effectively screen off those peptides that would not be stably MHC binding peptides in vivo.

Preamble to Examples of Stability Determination

The following example demonstrates one possible method for successfully obtaining data for stability between MHC molecules and peptides, which are presented by MHC molecules as a consequence of natural antigen processing in living cells. However, since the present invention demonstrates that such stability data provide for an important improvement of methods and tools for T-cell epitope prediction, the present invention is not limited to stability data acquired by means of the method below—reliable stability data obtained by any other method will provide the same improvement over existing T-cell epitope prediction methods and tools.

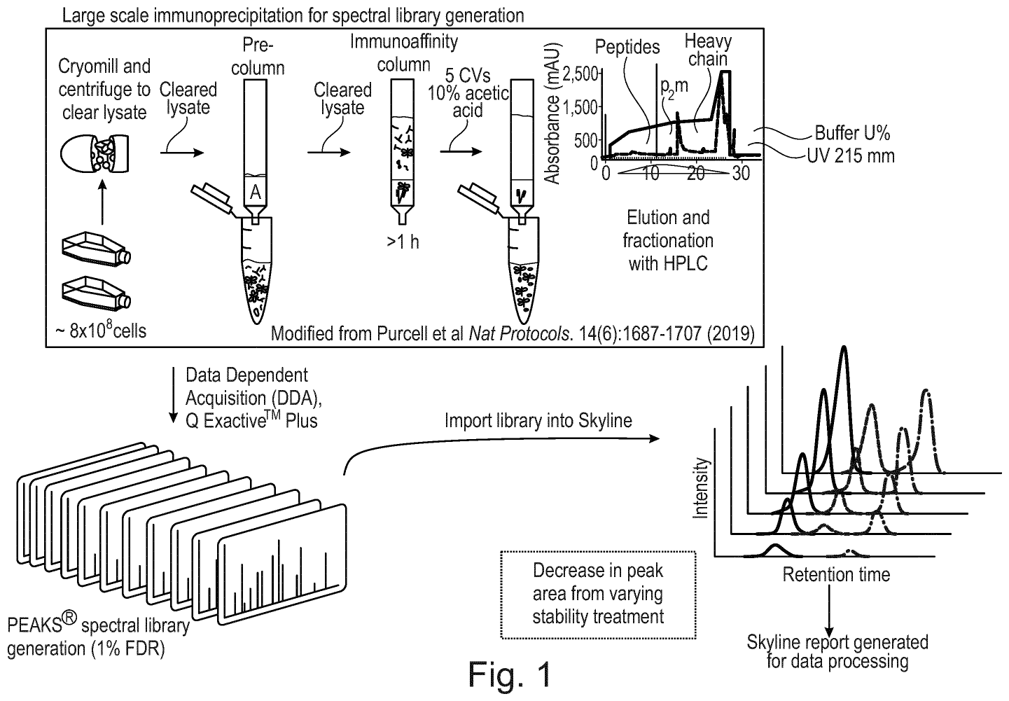

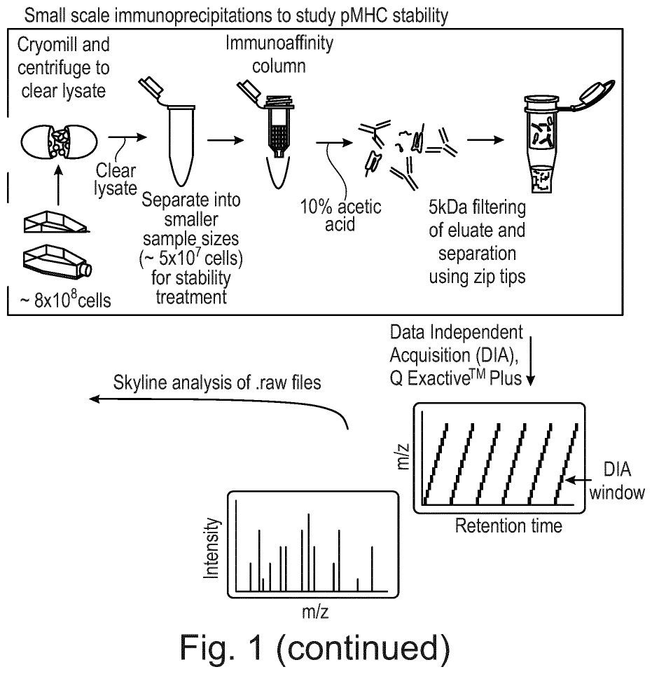

The present experiments were carried out using and expanding on the protocol of Purcell, Ramarathinam and Ternette, 2019 with certain modifications. The outline of the experiments performed below in the illustrative examples is set forth in the following and is also shown in schematic form in FIG. 1:
1) A mono-allelic cell line was prepared, cultured, and pelleted (in this case C1R cells were transfected to be mono-allelic for HLA-A*02:01).
2) Large scale immunoprecipitation/elution (cell pellet size≈8×10⁸ cells) was performed to create an MS spectral library as described in detail in the protocol of Purcell, Ramarathinam and Ternette, 2019.
3) Data dependent acquisition (DDA) mass spectrometry (MS) was performed using a Q Exactive (Thermo) on the large elution.
4) The PEAKS® software package was used to create a spectral library from DDA data for the MHC allele (in this case, the HLA allele) of interest.
5) Small-scale immunoprecipitation/elution (cell pellet sizes≈2×10⁷-5×10⁷) was performed on samples in triplicates or quadruplicates for each time/temperature point (cf. below) to create stability curves (time-course, exponential decay curves or thermal, sigmoidal curves)
6) Data independent acquisition (DIA) mass spectrometry (MS) was performed using a Q Exactive (Thermo) on all stability data replicates.
7) The Skyline software package was used to analyse and visualise peak areas of stability data replicates using the PEAKS®-generated spectral library to identify precursor and product ions.
8) MS peak areas (from step 6) of 8-mer-11-mer peptides were normalised based on peak areas of iRT peptides spiked into samples.
9) Peptides were filtered based on Skyline confidence threshold (dotP 0.85) with peak areas changed to 0 if peak confidence was less than the set threshold.
10) Peptides were filtered based on sequences from background peptides and unusual sequences.
11) Points were outlier corrected by calculating median of time/temperature point and neighbouring time/temperature points and taking mean of these median values.
12) Median intensity values for each peptide were fitted to a sigmoidal curve (for thermal stability measurements) or exponential decay curves (time course stability measurements) and, subsequently, thermal melting points (Tₘ) or half-life values (λ) were calculated, respectively.

### Example 1

Stability Determination: Large and Small Scale Immunoprecipitation/Elution

A mono-allelic cell line (C1R cells, mono-allelic for HLA-A*02:01) was grown, pelleted and stored at −80° C. for maximum 1 month.

The large scale protocol steps entailed

1) Crosslinking of antibody specific to the MHC molecule that it was desired to isolate (in the following experiments, antibody W6/32 was used) to Protein A Sepharose resin,

2) Grinding of large cell pellet (≈8×10⁸ cells) and clearing of lysate with centrifugation,

3) Addition of lysate to immune affinity column to isolate pMHC molecules of interest using w6/32 antibody,

4) Elution of pMHC molecules with 10% acetic acid,

5) Fractionation of sample and separation of peptides from the MHC molecule and β2m using HPLC (C18 clean-up of sample in preparation for MS),

6) MS analysis in DDA mode.

The small scale protocol steps entailed

1) Incubation of antibody specific to the MHC molecule that it was desired to isolate (in the following experiments, antibody W6/32 was used) to allow binding to Protein A Sepharose resin (1 hour incubation),

2) Grinding of large cell pellet (≈8×10⁸ cells) and separation of the lysate into sample replicates (≈5×10⁷ cells) for thermal or time course treatment,

3) Treatment of cell lysate with desired measure (heat or time) and subsequent cooling of the sample on ice for a few minutes,

4) Isolation of pMHC complexes using small columns (Mobispin®) with prepared antibody-resin,

5) Elution of pMHC complexes and antibody from resin using 10% acetic acid,

6) Separation of peptides from the larger molecules (MHC, β2m and antibody) using 5 kDa cut-off filters,

7) C18 clean-up of samples using zip tips, elution of sample in 0.1% formic acid, 30% acetonitrile, and

8) MS analysis on samples in DIA mode.

Reagents and Equipment for Antibody Cross-Linking Used in Large Scale Immunoprecipitation/Elution
Monoclonal antibody W6/32 (www.atcc.org/products/all/HB-95.aspx) specific for HLA-A*02:01 was used, either purified or from hybridoma supernatant. 10 mg of purified antibody per 1 ml of resin is needed, or approximately 1 litre of supernatant per 1 ml of resin depending on the hybrid. The isotype of the antibody was checked to determine whether it bound to protein A or G. Purified antibody or tissue culture supernatant were used to bind the resin. The amount of antibody in the supernatant generally ranged between 5-30 μg/ml depending on the B-cell hybrid.
Prior to doing a 10 mg coupling it was confirmed that DMP cross linking would not affect the binding capacity of the antibody. This was done using a small scale immunoprecipitation with antibody loaded beads with and without cross linking (as detailed at the end of this method). If this prevented the antibody from working a different resin such as NHS activated Sepharose® (the anti-HLA antibodies W6/32, L243, BB7.1, Rm5112, SPV-L3, B721, Y-3, 10.2.16 and 28.8.6s have previously been confirmed to work with DMP). For reference, antibody isotype and their affinities towards protein A and G are provided in the following table:

| Ig origin | Affinity for protein A | Affinity for protein G |
|---|---|---|
| Human IgG1, 2, 4 | +++ | +++ |
| Human IgD | − | − |
| Human IgA E, M | + | − |
| Human IgG3 | + | +++ |
| Mouse IgG1 | + | +++ |
| Mouse IgG2a, 2b, 3 | +++ | +++ |
| Mouse IgM | + | + |
| Rat IgG1 | + | + |
| Rat IgG2a | − | +++ |
| Rat IgG2b | − | + |
| Rat IgG2c | +++ | + |
| Bovine IgG1 | + | +++ |
| Bovine IgG2 | +++ | +++ |
| Chicken IgY | − | − |
| Dog IgG | +++ | + |
| Goat IgG1 | + | +++ |
| Goat IgG2 | +++ | +++ |
| Guinea pig IgG | +++ | + |
| Hamster | + | NA |
| Horse IgG | + | +++ |
| Monkey IgG | +++ | +++ |
| Porcine IgG | +++ | +++ |
| Rabbit IgG | +++ | +++ |
| Sheep IgG1 | + | +++ |
| Sheep IgG2 | +++ | +++ |

Poly-Prep Chromatography column (BioRad), yellow top and bottom caps.
Tubing and syringes.
10% acetic acid in fresh MilliQ (mass spec grade acid in new glassware that has not been washed with detergent but rinsed with MilliQ).
Protein A Sepharose® Fast Flow (PAS; Amersham).
PBS (phosphate-buffered saline), filtered.
Borate Buffer:
   Solution “A”: 0.1 M Boric acid/0.1 M KCl
      Per 100 ml
         boric acid: (Mw 61.83 g): 0.62 g.
         KCl (Mw 74.56 g) 0.75 g.
   Solution B: 0.1 M NaOH (Mw 40.00 g) 0.4 g/100 ml.
   For 100 ml of Borate buffer pH 8:
   50 ml A+3.97 ml B+46.03 ml fresh MQ, checked for pH=8 with pH paper, and filtered through 0.2 μM PES filter.
Tris: 0.2 M, pH 8, filtered, kept cold.
Citrate: 0.1 M, pH 3, filtered.
Triethanolamine (TeO; stock density 1.125 g/ml, Mw 149.19 ie 7.54 M)
   Viscous solution, use cut blue tip.
   1.326 ml TeO per 50 ml, adjust pH to 8.2 with HCl, not filtered.
   Dimethyl pimelimidate (DMP; Sigma D8388): 40 mM in 0.2 M Triethanolamine. DMP is prepared by dissolving 250 mg (1 vial) DMP-2HCl in 22 ml 0.2 M Triethanolamine pH 8.2. pH is adjusted to 8.3 with NaOH, and brought to 24.1 ml, without filtering. DMP solutions should be prepared and used on the same day. Generally one 250 mg vial is used per 2 ml resin.
Retort stand

Procedure for Antibody Cross-Linking in Large Scale Protocol
1. A cap was placed on the bottom of the column and filled with 10% acetic acid and allowed to sit for 20 min at room temperature. The cap was removed and the column allowed to flow through, rinsed with a further 10 ml of acid, and then thoroughly with milliQ water in order to extract any non-adhering polymer.
2. PAS was fully resuspended in a bottle and the required amount removed using a 1 ml tip. The protein A Sepharose® (PAS) was supplied as a ˜50% slurry (confirmed by visual inspection before resuspending), therefore for every 1 ml of bed volume, 2 ml of slurry was required (the calculation was adjusted if slurry deviated from 50% resin).
3. PAS was added to a column with bottom cap in place and allowed to settle, and subsequently washed with 10 column volumes (CV) PBS.
   Flowrate through the column was if necessary increased by attaching thoroughly cleaned tubing to the top of the column and the other end to the barrel of a 50 ml syringe secured as high as practical above the column, filling the syringe with PBS, removing the bottom cap, and washing the resin by gravity flow with 10 CV PBS. Alternatively, the flowrate was increased by attaching tubing to the top of the column and the other end to the barrel of a 50 ml syringe, and slowly depressing the plunger to create back pressure on the column to ensure that the drop rate through the column did not exceed 1 drop per second.
4. 10 mg of antibody was bound per 1 ml resin by batch i.e. using a 1 ml pipette, the PBS washed resin was removed from the column and placed in 50 ml tube. The purified antibody was purified to ˜15 ml with PBS, added to the resin and rotated end over end in a cold room for 30-60 min.
5. The resin was loaded back into the column at room temperature using borate buffer to wash out the interior of the 50 ml tube and recover all the resin. If antibody containing supernatant was used, it was after step 3 loaded straight onto a washed column in the cold room (after supernatant was loaded, the procedures typically proceeded at RT). When using antibody-containing supernatant, it was also determined how much antibody the relevant hybrid was secreting, and it was tested that the supernatant contained specific antibody. If the secretion turned out to be low (less than 5 μg/ml) the hybrids were re-cloned.
   (If purified antibody was used, a sample taken from the starting material added to the resin in step 5 and a sample of the flow through (i.e. step 6) (25 μl sample+25 μl sample buffer) were compared to make sure the flow through was fairly well depleted).
6. A wash with 10 CV borate buffer pH 8 was carried out.
   For testing: After washing, 25 μl aliquot of beads were placed into Eppendorf tubes by resuspending the beads at the top of the column and adding 25 μl reducing SDS sample buffer. At this point the antibody were not covalently bound to the beads, so when the sample was boiled in reducing sample buffer, the antibody disassociated from the beads and the heavy and light chains become clearly visible by Coomassie staining (approx 50 kDa and 25 kDa).
7. A wash with 10 CV freshly-made 0.2 M triethanolamine, pH 8.2 was carried out to equilibrate the column. The use of triethanolamine ensures that no free amines are present in the buffer system as this could interfere with the efficiency of crosslinking by DMP to primary amines in the protein A bound antibody.
8. Cross-linking was carried out by passing ˜25 ml of 40 mM DMP in 0.2 M triethanolamine over the column, halting the flow leaving a meniscus covering the resin, then leaving at room temperature for 1 hr. This amount of DMP is sufficient for at least 20 mg of antibody and can stretch to 30 mg.
9. The cross-linking reaction was terminated by flowing over 10 CV of ice-cold 0.2 M Tris pH 8.
10. A wash was carried out with 10 CV 0.1 M citrate buffer pH 3 and collect flow through. The citrate wash will strip any antibody that has not been covalently linked.
   For testing: After washing in citrate, 25 μl aliquot2 of beads were mixed with 25 μl reducing SDS sample buffer. As the antibody was covalently cross-linked it remained attached to the beads even after boiling in SDS sample buffer (although generally there a small amount of leeching was observed).
11. A wash was carried out with 10 CV 0.1 M borate buffer (or PBS) with 0.02% NaN₃, pH 8, for storage at 4° C.
12. The flow through from step 9 was concentrated down to 500 μl using a 15-30 kDa cut off Millipore concentrator. To monitor the cross linking reaction, a 12% SDS PAGE gel was run for Coommassie staining as follows:
   1. unstained protein ladder
   2. 25 μl beads (step 5)+25 μl reducing SDS SB; boil, run 20 μl.
   3. 25 μl beads (step 9)+25 μl reducing SDS SB; boil, run 20 μl.
   4. 25 μl conc flow through (step 11)+25 μl reducing SDS SB; boil, run 20 μl.
      This demonstrated the presence of antibody in sample 2 (before cross-linking) but not in sample 3 (after cross-linking, although there may be a small amount) and no or only a little in sample 4 (concentrated citric acid strip post cross-linking).

Reagents for Large Scale Immunoprecipitation
10% IGEPAL 630 (Sigma) stock in MilliQ (protected from light) 1M Tris pH 8
2M NaCl
10% acetic acid (mass spec grade).
Total protease inhibitor cocktail (Roche): 1 tablet is enough for 50 ml buffer, if less than 50 ml is required, make 25× stock by dissolving 1 tablet in 2 ml fresh MilliQ water, aliquot and store at −20° C. up to 4 months.
Protein A Affinity Resin
Poly-Prep Chromatography column (BioRad) for preparation of pre-column (if the column has not previously been used for peptide elution, place cap on bottom of column and fill with 10% acetic acid, sit for 20 min at RT, remove cap and allow to flow through, rinse with a further 10 ml of acid followed by MilliQ).
For preparation of 1× lysis buffer (for small cell pellets<4×10⁸ cells):
   0.5% IGEPAL 630
   50 mM Tris, pH 8.0
   150 mM NaCl
   1× total protease inhibitor cocktail
   MilliQ water (make sure this is freshly drawn)
For preparation of 2× lysis buffer (for large cell pellets>4×10⁸ cells):
   1% IGEPAL 630
   100 mM Tris, pH 8.0
   300 mM NaCl
   2× total protease inhibitor cocktail
   MilliQ water (make sure this is freshly drawn)
This buffer was adjusted to 1× after cell grinding to accommodate the volume of the cells.
Ultracentrifuge tubes (only required for cell pellets>4×10⁸ cells); polycarbonate 26.3 ml capacity.
Washbuffer 1:
   0.005% IGEPAL
   50 mM Tris, pH 8.0
   150 mM NaCl
   5 mM EDTA
   100 μM PMSF (0.1 M stock in Abs EtOH; stored at −20° C.)
   1 μg/ml Pepstatin A (1 mg/ml stock in isopropanol; stored at −20° C.)
   In MilliQ H₂O
   Filtered through 0.2 μM syringe filter, keep on ice.
Washbuffer 2:
   50 mM Tris, pH 8.0
   150 mM NaCl
   in MilliQ H₂O
   Filter through 0.2 μM syringe filter, keep on ice.
Washbuffer 3:
   50 mM Tris, pH 8.0
   450 mM NaCl
   in MilliQ H₂O
   Filter through 0.2 uM syringe filter, keep on ice.
Washbuffer 4:
   50 mM Tris, pH 8.0
   in MilliQ H₂O
   Filter through 0.2 uM syringe filter, keep on ice.
Retort stand

Procedure for Large Scale Immunoprecipitation and Peptide Elution

When small cell pellets were prepared, 1× lysis buffer was used and the ultracentrifugation step was replaced with centrifugation of lysates in a microcentrifuge at 13000 rpm for 20 min at 4° C. Column loading, washing and elution should be performed in cold room.
1. Cells were lysed in lysis buffer at approx 1.25×10⁸ cells per ml.
2. The frozen cell pellets were in each case ground in a cryogenic mill according to the following procedure:
   A foam dewar was filled with liquid nitrogen in a fumehood
   A 10 ml container was pre-cooled with one 10 mm ball in nitrogen bath.
   The Cell pellet was dislodged from the base of the tube by tapping on the workbench.
   If the pellet was large, it was dissected into pea sized pieces with a scalpel blade.
   1-2 pieces of the pellet were placed in the 10 ml container with ball, placed back in nitrogen to cool again and then placed in the cryogenic mill ensuring the other side was balanced with a second 10 ml container in the same position.
   The cells were ground 30 Hz for 1 min, removed and checked to ensure that the material appeared like a fine powder, scraped out and placed directly into a tube containing cooled lysis buffer.
3. Step 2 was repeated with remaining pieces.
4. When all material was transferred to the lysis buffer, the volume was adjusted to 1× using fresh MilliQ water and incubated in the cold room rotating end over end for 45 min.
5. While sample was lysing, columns were prepared in cold room:
   A 0.5 ml pre-column was prepared by placing 1 ml protein A slurry into a Poly-Prep column, washed with 10 CV of 50 mM Tris pH8 (wash buffer 4) to remove ethanol, then equilibrated with 10 CV of wash buffer 1, and capped at the bottom.
   The affinity column was set up, equilibrated with 10 CV wash buffer 1, and capped in the bottom.
6. Lysate was centrifuged for 10 min at 4000 rpm at 4° C. to remove nuclei.
7. Supernatant was transferred into a pre-chilled ultracentrifuge tube filled almost to the top (if necessary with addition of further lysis buffer) and centrifuged in a Ti70 rotor for 45 min at 40,000 rpm, 4° C.
8. Supernatant was collected into pre-cooled 50 ml tubes. The supernatant should be clear, but if there remained layer of lipid on the top, this was removed carefully with a 1 ml filter tip and kept on ice in a separate tube.
9. Supernatant was run over the pre-column and collected in a 50 ml tube and then transferred onto the affinity column or the columns were set up in tandem to let the flow-through drip directly from the pre-column to the affinity column. The lysate was put over the affinity column without tubing for the first pass to ensure a slow passage over the column. The flow-through was collected.
10. The lysate was run over the affinity column two more times by attaching clean tubing to the top of the column and loading from a good height above the column from a 50 ml tube to ensure a quicker flow and allowing the lysate to be passed over multiple times.
11. The column was washed with 20 CV of cold wash buffer, with 20 CV of cold wash buffer 2 (to remove detergent), with 20 CV of cold wash buffer 3 (to remove non-specifically bound material), and finally with 20 CV of cold wash buffer 4 (to remove salt to prevent crystal formation).
12. It was ensured that the meniscus was just above the resin. All tubing was removed, and the column was eluted using 5 CV of 10% acetic acid by using either a 1 ml filter tip or a clean glass 10 ml cylinder. The eluate was collected into a clean 25 ml glass beaker or into as 2 ml low-bind Eppendorf tubes.

Reagents and Equipment for MS Ligand Small Scale Experiment with Stability Testing (Time Scale Varied and Temperature Varied)
Cells that had been pelleted at snap-frozen, cf. above (cell pellet size 2×10⁷-5×10⁷)
Protein A Sepharose Fast Flow (PAS; Amersham)
Monoclonal antibody either purified or supernatant. Need 2 mg of purified antibody per 1 ml of Protein A resin
20% IGEPAL 630 (Sigma) stock in MilliQ (protect from light) 1M Tris pH 8
5 M NaCl
10% acetic acid (mass spec grade, tested for purity)
Total protease inhibitor cocktail (Roche): 1 tablet is enough for 25 ml buffer, if less than 25 ml is required, make stock by dissolving 1 tablet in 2 ml MS grade water, aliquot and store at −20° C. for up to 4 months.
For preparation of 1× lysis buffer (25 ml total volume), 500 μl lysis buffer needed for lysis of 5e7 cells:
   0.5% IGEPAL 630 (0.625 ml 20% IGEPAL630)
   50 mM Tris, pH 8.0 (1.25 ml 1M Tris, pH 8.0)
   150 mM NaCl (0.75 ml 5M NaCl)
   1× total protease inhibitor cocktail (2 ml of 25× stock (1 tablet dissolved in 2 ml MS grade water)
   MS grade water (20.375 ml to make up total of 25 ml)
Filtered 1×PBS
MobiSpin® columns (www.mobitec.com/cms/products/bio/10_lab_suppl/mobicols2.html)
Low-bind 2 ml Eppendorf tubes
Centrifugal filter units (Merck Millipore)
Pipettes and tips (Eppendorf)
Beaker for chemical waste
Heat block
Timer
Fridge at 4° C.
Ice to keep lysis buffer and PBS cold
Sterile hood for preparation of resin and antibody
50 ml tubes for incubating Eppendorf tubes with samples
Sample roller at 4° C.
Table-top centrifuge
Table-top ForceMini spinner to pulse-spin samples

Procedure Ligand Stability Testing (Time Course and Thermostability)

Day 1
1. 1×PBS (sterile) was prepared from a 10× stock
2. 1× affinity columns (MobiSpin®) were prepared for each sample:
   2 ml Eppendorf tubes (one for each column) were prepared by clipping off the lid (discard the lid)
   All columns were uncapped and placed in Eppendorf tubes
   All columns were washed with 2×550 μl of 10% acetic acid, the columns were sealed and pulse-spun 8-10 sec on ForceMini between washes, and the acetic acid was discarded
   All columns were washed with 2×550 μl PBS, and spun (with the lid tightened) 8-10 sec between each wash
3. Protein A resin was prepared in columns and antibody was coupled to protein A resin:
   Antibody was bound at a ratio of 400 μg to 200 μl (2 mg/ml in comparison to 10 mg/ml when performing large scale elutions) of Protein A resin
   200 μL of Protein A resin was added to the affinity columns, which equates to 400 μL protein A-ethanol slurry (assuming 1:1 ratio)
   After adding protein A resin to each column, they were spun to remove ethanol (5-10 sec) and the ethanol discarded
   The columns were washed 3× with PBS (to max volume, ˜500 μl of PBS) and the PBS was discarded
   All columns were capped and ˜150 μL PBS were added to the columns to avoid drying
   For the affinity column, antibody was under sterile conditions added to a 2 ml Eppendorf tube at the required volume to add 400 μg and used to transfer the washed resin from the column into the tube. It was ensured that all resin had been transferred by using additional PBS. The Eppendorf tubes were placed in a 50 ml tube and incubated at 4° C. for at least 1 hr with gentle rotation
   The ‘empty’ affinity columns were left on ice (capped and with lid)
4. Cells were lysed as follows:
   The heat block was switched on at appropriate temperatures for incubation of lysate (37° C. for the time scale experiments, a range of temperatures for the thermostability experiments).
   Set centrifuge (for 50 mL tubes) to 4° C. (13,000 rpm, 10 mins) and centrifuge for Eppendorf tubes to 4° C. (13,000 rpm, 10 mins)
500 uL lysis buffer per 5e7 cells was prepared and kept on ice:
Grind cell pellet if >4e8 cells using cryogenic mill
   a. The foam dewar was filled with liquid nitrogen. The container was precooled with one 10 mm ball and on 7 mm ball in the nitrogen bath.
   b. The cell pellet was dislodged from the 50 mL tube and transferred to the pre-cooled container.
   c. The container was balanced with a second container. The cell pellet was smashed at 30 Hz for the appropriate amount of time to make powder (5-90 mins), removed and checked during grinding.
   d. The ground cells were transferred to the appropriate amount of lysis buffer (in 50 mL tube).
   e. If pellets are small, lysis buffer is added directly to cell pellet(s); 500 uL lysis buffer per 5e7 cells and gently resuspend pellet with pipette until thawed/dissolved
   Leave to lyse at 4° C., 45 min, rolling
5. Lysate was centrifuged and the lysate supernatant was added to the affinity column
   Clear lysate by spinning for 10 mins at 13,000 rpm.
   Transfer lysates to 2 mL Eppendorf tubes to make up the desired number of sample replicates and spin for 10 mins at 13,000 rpm
   The lysate supernatant was added to a new 2 ml Eppendorf tube and placed on a heat block. For the time course stability experiment, the lysate was incubated at 37° C. for either 0, 0.5, 1, 1.5, 2, 3, 5 or 24 hours (in desired number of replicates). For the thermal stability experiment the lysate was incubated for 10 mins at either 37° C., 40° C., 43° C., 46° C., 50° C., 53° C., 56° C., 60° C., 63° C., 66° C., 70° C., 73° C. (in desired number of replicates).
   Upon completion of the incubation, the Eppendorf tubes were put straight on ice.
6. Treated lysate was added to affinity column with washed antibody-resin
   The antibody-resin mix was transferred back to the column and spun through the column. The antibody-resin column was then washed thoroughly, 3× with PBS (550 μl) and resuspended between washes. The columns were capped and a small volume of PBS was if necessary added to the Ab-resin beads to avoid them going dry.
   The lysate was added (300-400 μl at a time) to the washed Ab-resin mixture in the affinity Mobispin column (capped), resuspended and transferred back to the lysate Eppendorf tube. Any residual resin beads in the column were transferred using additional PBS (100-200 μl). The Eppendorf tubes were each placed in a 50 ml tube to incubate and rotate at 4° C. overnight.

Day 2
1. Centrifugal filter units (Merck Millipore) were prepared
   Filter units were washed with 500 μl of 10% acetic acid ×2; spun at RT, 13,000 rpm for 60 mins after adding 10% acetic acid, and removal of the acid after spin
2. Antibody-bound molecules were eluted from protein A resin (after overnight incubation) according to the following steps:
   1×PBS (sterile) was prepared from 10× stock and kept on ice
   Resin with bound antibody and lysate was transferred from the overnight incubated Eppendorf tubes to the affinity columns saved from the day before
   Uncapped columns were pulse-spun for 8-10 s.
   The affinity column was washed with PBS (550 μl) ×3 (up to ×5), spun between each wash and the flow-through was discarded.
   New Eppendorf tubes were prepared (without cutting off the lid) for the eluate and elution was carried out using 10% acetic acid ×4 rounds of 100 μl; in each round the elution was carried out for 5 mins for a slower flow-through, then the Eppendorf tubes were spun for 5-7 sec, and the flow-through was saved for each of the elution rounds (total 400 μl eluate).
   The eluate was heated to 70° C. (˜10 mins), then cooled to RT (˜2-3 mins) before loading these onto the filter
3. Loading samples onto the filter units
   1.5 ml Eppendorf tubes were prepared for the flow-through from the filter
   Once filter units had been washed, the lid was cut off and the bottom part was discarded while saving the filter and the lid
   The filter was placed in the new Eppendorf tubes, the pre-heated acetic acid eluate was added and the lid placed on the filter to ensure a tight closure
   Samples were spun at RT, 13,000 rpm, for at least 30 mins until all sample has passed through the filter
   Buffers were prepared in 50 ml tubes for zip tipping
   After the spin to filter the eluate, the filter was washed with 200 μl zip tip buffer A (0.1% formic acid) by spinning 13,000 rpm for approx. 30 mins (or more, ensuring that all of buffer A had passed through the filter) to allow for any remaining/additional peptides to come off the filter into a new Eppendorf.
4. Eppendorf tubes with flow-through from the filter units (peptides) are stored in the fridge until zip-tip protocol is carried out.

Zip-Tip Protocol for Small Scale Samples

Reagents and Materials:
Buffer A: 0.1% formic acid in MS-grade water
   For 1 ml: 999 μL water+1 μL formic acid
Buffer: 0.1% formic acid in 30% acetonitrile (v/v) in MS-grade water
   For 1 ml: 300 μl acetonitrile, 699 water+1 μl formic acid
Eluted peptide samples
iRT peptides (200 fmoles per sample)—www.biognosys.com/shop/irt-kit
Low-bind Eppendorf tubes (1.5 ml)
Zip tips (100 μl)
50 ml falcon tubes for buffers
Pipettes and tips
Beaker for chemical waste
Speedy Vac

Procedure
iRT peptides were taken from −80° C. freezer and spiked in at 200 fmoles of iRTs per sample
Zip-tip buffers were prepared
200 μl zip-tip was pre-wetted 3 times with 100 μl of buffer B
Equilibration was carried out 3 times with 100 μl of buffer A
Sample was bound by pipetting up 100-200 μl sample, transferring to new Eppendorf tubes, pipetting up and down several times until all sample has been bound
3 washes with 100 μl buffer A was carried out
3 time elution was carried out with 100 μl buffer B
Samples were dried until almost completely dry on speedy vac (300 μl samples ˜1-2 hours)
Samples were reconstituted in 0.1% formic acid, 2% ACN, sonicated and spun down
The desired volume (10-20 μl) was transferred to an MS vial
MS samples were run

MS Analysis of Eluted Peptides

The large scale eluted peptides were separated by means of RP-HPLC and subjected to LC-MS/MS analysis according to the protocol described in Purcell et al. 2019. The PEAKS® software package was used to create a spectral library from data dependent acquisition (DDA) data generated based on the large scale elution fractions for a specific HLA allele. The small scale samples which had been subjected to incubation at different temperatures/times as described in the protocol and cleaned up using the described zip tip protocol were subjected to LS-MS/MS in data independent acquisition (DIA) mode. In this case, both DDA and DIA MS were performed using a Q Exactive (Thermo).

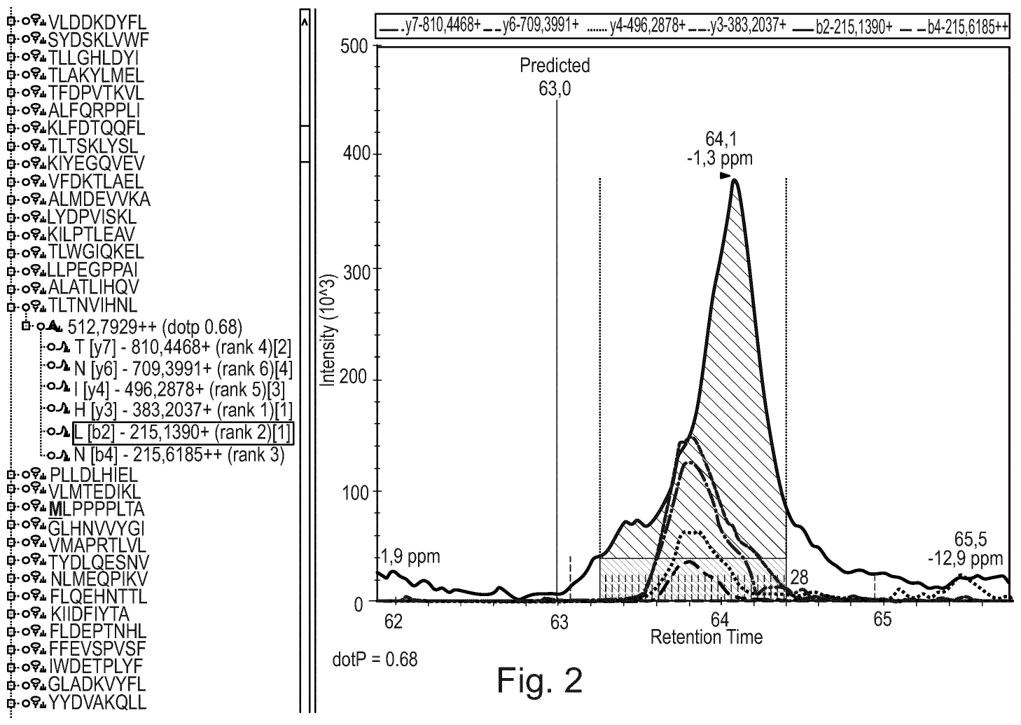

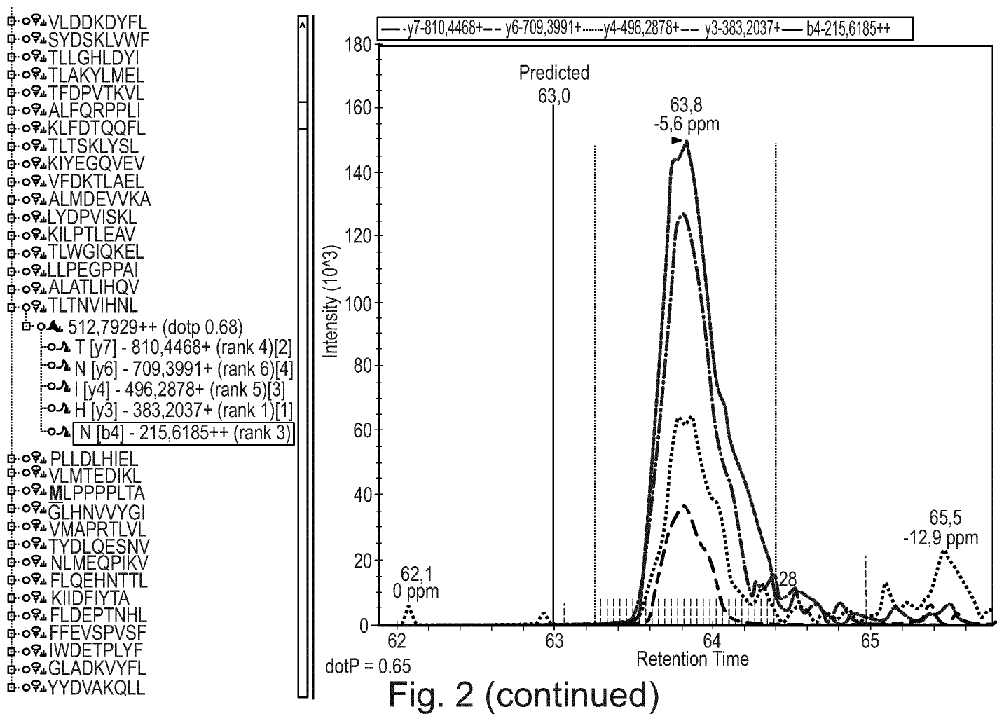

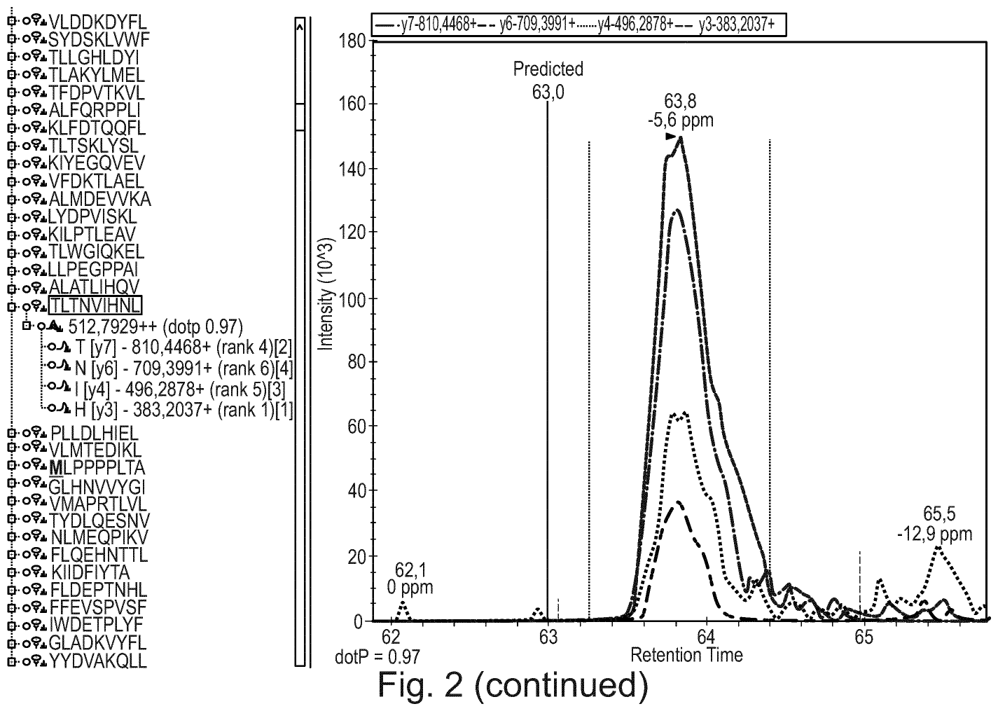

Subsequently, the Skyline software package was used to analyse and visualise peak areas of stability data replicates using the PEAKS®-generated spectral library to identify precursor and product ions (see FIG. 2 for an example).

All 8mer-11mer peptide peak areas were normalised based on iRT peptides spiked into the samples: a) The weighted iRT values were calculated: Each individual iRT values was divided by the mean of the iRT values for the given iRT peptide across replicates and 2) the mean value for each replicate was then calculated across all weighted iRT values for a given replicate:

$$Normalized value = \frac{1}{J} {\sum }_{j} \frac{{x}_{ij}}{\frac{1}{I} {\sum }_{i} {x}_{ij}} Corrected ligand intensity = Ligand intensity / Normalized value$$

Peptides were now filtered based on a Skyline confidence threshold (dotP 0.85) for the median value of the 37° C. samples with peak areas set to 0 if peak confidence was less than the set threshold. Finally, the peptides were filtered based on sequences from background peptides (sequences from protein digests of the HeLa cell lines as well as sequence motifs indicating peptide binding to HLA-C*04:01 and HLA-B*35:05 which are naturally presented on parental C1R cells that have been transfected with the HLA of interest) and unusual contaminant sequences (often sequences with multiple prolines adjacent to one another). In the thermostability test series, this approach resulted in data for 491 peptides (8mers-11mers) from the different temperatures tested. In the time course experiment, the same approach provided data for 353 peptides (8mers-11mers).

For the thermostability experiment, points were outlier corrected by calculating the median of a temperature point and neighbouring temperature points and selecting the mean of these median values. Then, the median intensity values fitted to a sigmoidal curve

$$f ( T ) = \frac{1}{1 + {e}^{s ( T - {T}_{m} )}}$$

where s is the slope of the linear part of the fitted sigmoidal curve and Tₘ is the melting temperature.

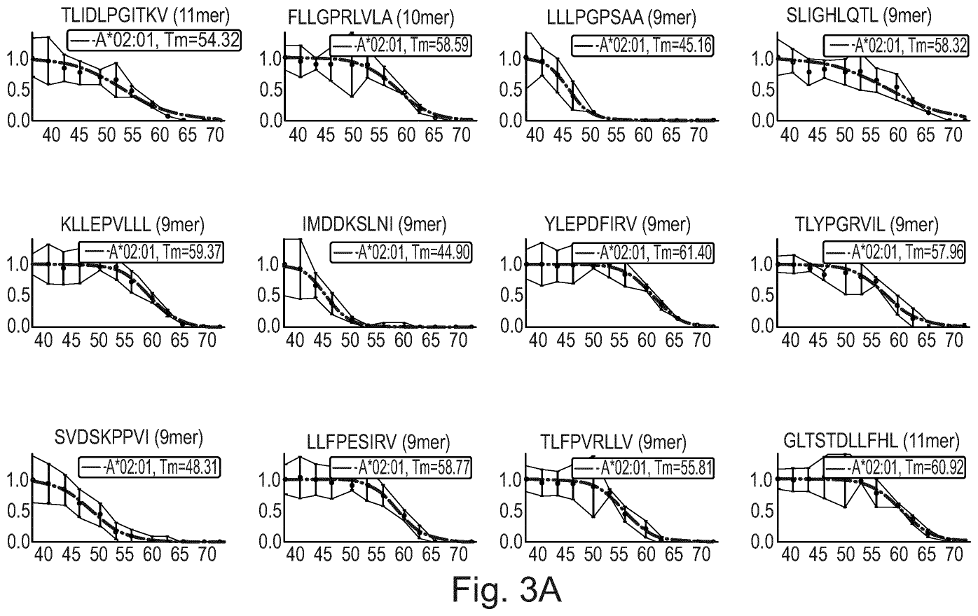

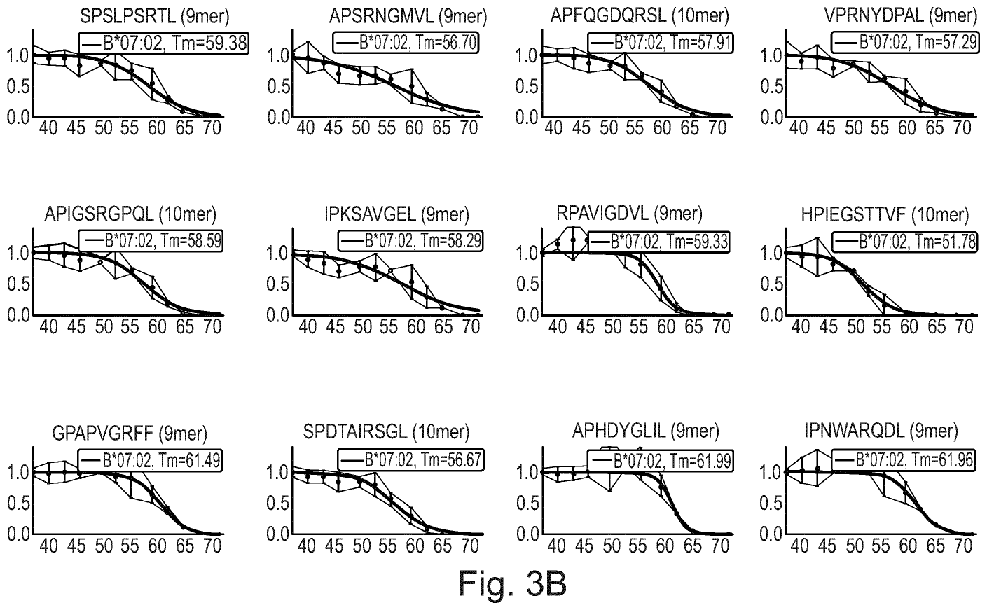

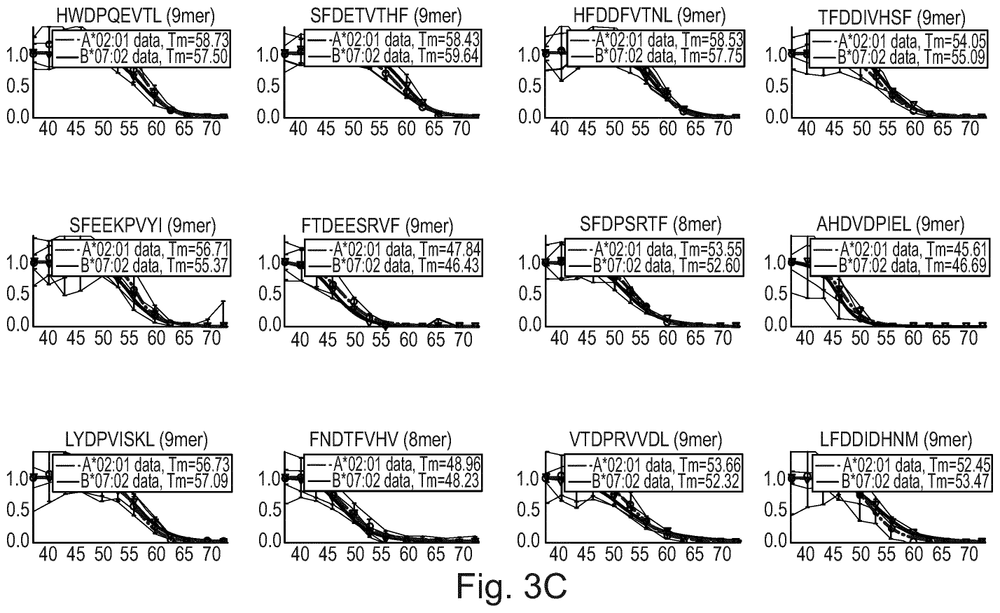

Examples of melting point determinations from fitted sigmoidal curves are provided in FIG. 3.

For the time course stability experiment, the median intensity values were fitted to an exponential decay curve

f(x)=e^{-Kx}

which indicates that the value of f(x) at the initial time point (time zero) is 1 and the exponential decay curve approaches the value f(x)=0 asymptotically. K is the rate constant from which the half-life of the complex can be calculated as follows

$$λ = \frac{ln ( 2 )}{K}$$

Finally, the determined 491 melting points were subjected to linear normalization to arrive at melting point values arbitrarily set to values between 0.5-1.0 by calculating a normalized value for each of the Tₘ values:

$${T}_{m normalised} = 0.5 \cdot \frac{{T}_{m} - min ( {T}_{{m}_{a𝔩𝔩}} )}{max ( {T}_{{m}_{all}} ) - min ( {T}_{{m}_{a𝔩𝔩}} )} + 0.5$$

These normalised Tₘ values allowed a simple ranking of the peptides with respect to their relative melting points. See FIG. 4, which in the left-hand panel shows—in a bar graph format—the distribution of the normalized Tₘ values and their frequencies, and which in the right-hand panel shows the information available if not performing a thermal stability determination. If solely relying on the data available in the right-hand graph, all 491 peptides would be considered equally useful ligands for HLA-A*02:01, whereas the left-hand panel bar graph demonstrate that only about 40% of the 491 peptides appeared in the group of peptides with high thermostability.

### SUMMARY OF RESULTS

A novel assay was established. The assay combines thermal/time-course treatment of cell lysates with mass spectrometry, cf. FIG. 1.

Peptides were filtered using PEAKS® and Skyline software packages, with the latter software being used for peak picking, cf. FIG. 2.

The assay was successfully used to generate MS data that can be transformed into stability values for the HLA ligands present in the treated peptide samples from cells being mono-allelic for HLA, see FIG. 3, which depicts the thermal stability curves for a number of peptides identified and quantified according to the presently presented method.

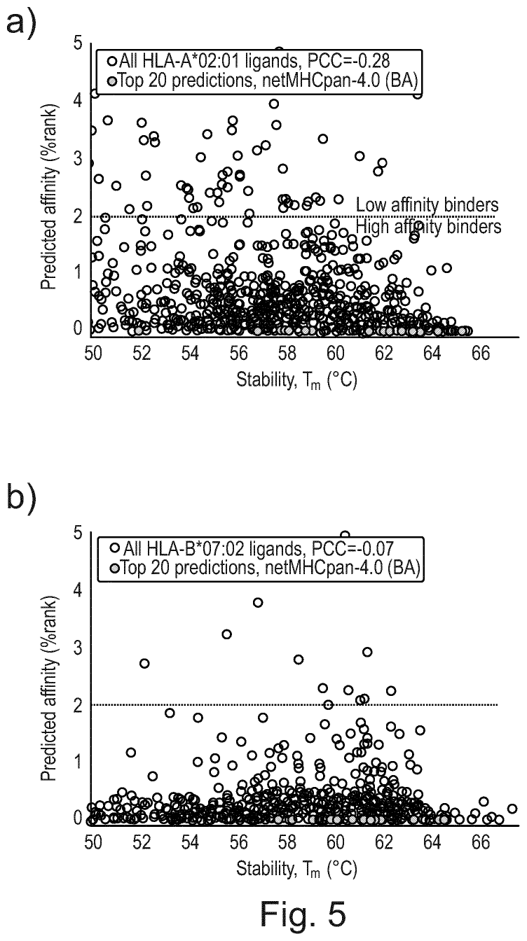

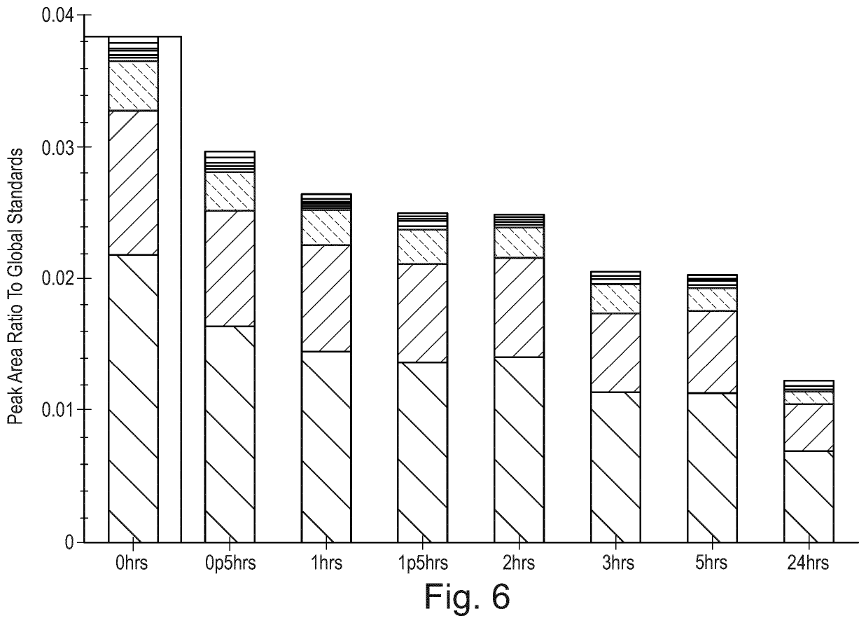

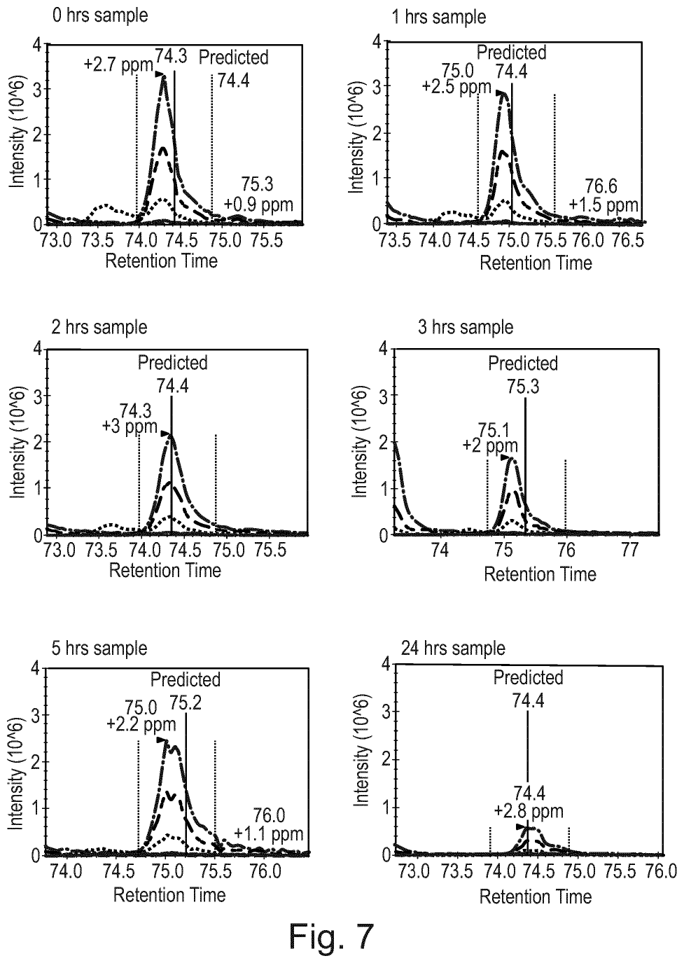

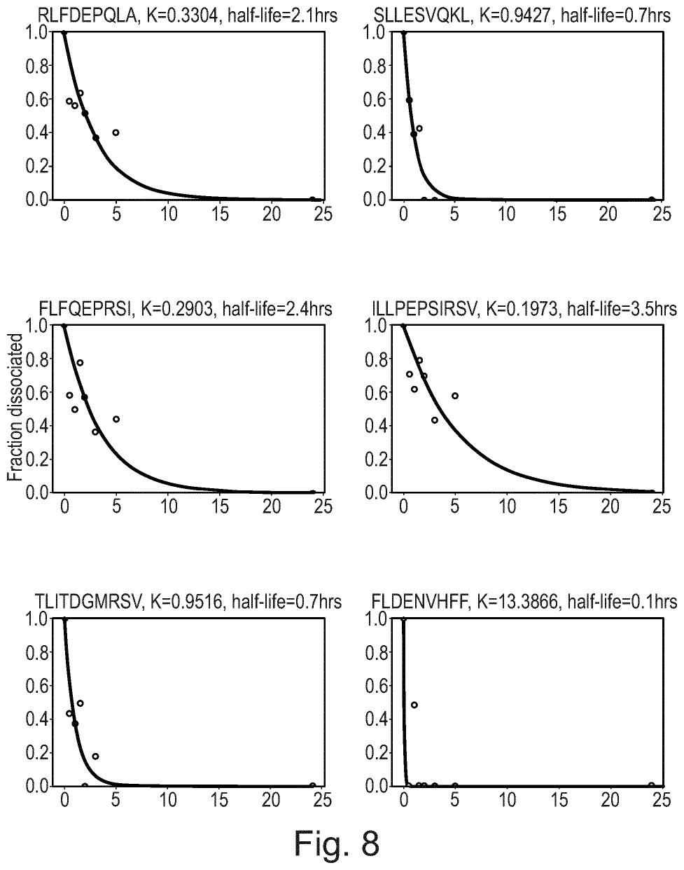

It was in addition investigated whether there is correlation between predicted ligand rank score (netMHCpan4.0, cf. www.cbs.dtu.dk/services/NetMHCpan/) and the determined thermal stability values for the HLA ligands, see FIG. 5. From this figure it is clear that a large number of high stability peptides are not predicted by the existing ligand rank score software and also that some peptides predicted in practice were demonstrated to be very poor ligands having low thermostability.

To summarize, the present technology enables an enhanced MHC ligand determination, which in turn makes it possible to rationally design peptide based vaccines to 1) avoid inclusion of peptides, which—although they are ligands for MHC molecules—have too low stability to be relevant as T-cell immunogens, 2) allow inclusion of peptides which all exhibit the desired stability (typically high or intermediate) for MHC binding. The presently disclosed quantitative measure for pMHC binding (pMHC stability) can be importantly be incorporated into current prediction algorithms to improve the prediction of T cell epitopes.

One important feature in this respect is that the method allows that the stability of binding is investigated at near-physiological temperatures, whereas previously applied methods for identifying naturally processed peptides have been carried out at non-physiologically low temperatures (in Purcell et al. 2019, the complexes of MHC molecules and peptides are e.g. at no point subjected to temperatures>4° C., but the complexes were naturally presented by the cells at physiological conditions prior to the steps taken to isolation and elution). In particular, the present approach of applying a time-course treatment provides, when carried out at temperatures≈37° C., information about the stability (and in particular the lack of stability) of binding between peptides and MHC molecules that are found to be stably bound in vitro at low temperatures.

In addition, the fact that only peptides eluted from cells that have naturally processed proteins comprising the peptides means that the identified peptides are inherently verified as being products of antigen processing:
The assay assesses the ‘true’ off-rate, as peptides have already bound to the MHC complex within the cell as part of the natural antigen processing and presentation;
the competition for binding to MHC between peptides in the natural cell environment is inherently part of the inventive assay, whereas traditional pMHC affinity assays gauge competition for MHC binding between a peptide and a labelled competitor in an isolated manner;
processing of antigens via the antigen processing machinery is naturally incorporated; and the assay minimises bias as it does not require pre-selection of peptides for analysis—the cell has naturally selected the peptides via its intracellular machinery.

Furthermore, the method developed is readily applicable on all MHC expressing cells, in particular all mono-allelic cell lines and the method is not restricted by the ability to re-fold MHC heavy chain and β2m in vitro.

The natural cell setting that this method is built upon results in features such as affinity and antigen processing being anchored in the assay. Furthermore, the natural cell setting avoids the bias that other stability assays are prone to. Bias in other assays mainly results from the fact that many peptides are selected for synthesis based on prior knowledge from other studies that have investigated epitopes or based on affinity prediction models resulting in circular reasoning potentially becoming an issue.

### LIST OF REFERENCES

Blaha, D. T. et al. (2019) ‘High-Throughput Stability Screening of Neoantigen/HLA Complexes Improves Immunogenicity Predictions’, Cancer Immunol Res 7(1): 50-62. doi: 10.1158/2326-6066.CIR-18-0395.
Gfeller, D. et al. (2016) ‘Current tools for predicting cancer-specific T cell immunity’, OncoImmunology 5(7): 1-9. doi: 10.1080/2162402X.2016.1177691.
Harndahl, M. et al. (2012) ‘Peptide-MHC class I stability is a better predictor than peptide affinity of CTL immunogenicity’, Eur J Immunol 42(6): 1405-1416. doi: 10.1002/eji.201141774.
Jørgensen, K. W. and Buus, S. (2014) ‘NetMHCstab—predicting stability of peptide—MHC-I complexes; impacts for cytotoxic T lymphocyte epitope discovery’, Immunology 141(1): 18-26. doi: 10.1111/imm.12160.
Koşaloğlu-Yalçin, Z. et al. (2018) ‘Predicting T cell recognition of MHC class I restricted neoepitopes’, OncoImmunology 7(11): 1-15. doi: 10.1080/2162402X.2018.1492508.
Mei, S. et al. (2019) ‘A comprehensive review and performance evaluation of bioinformatics tools for HLA class I peptide-binding prediction’, Briefings in Bioinformatics: 1-17. doi: 10.1093/bib/bbz051 (Epub ahead of print).
Purcell, A. W., Ramarathinam, S. H. and Ternette, N. (2019) ‘Mass spectrometry-based identification of MHC-bound peptides for immunopeptidomics’, Nature Protocols. 14(6): 1687-1707. doi: 10.1038/s41596-019-0133-y.
Rasmussen, M. et al. (2016) ‘Pan-Specific Prediction of Peptide-MHC Class I Complex Stability, a Correlate of T Cell Immunogenicity’, J Immunol 197(4): 1517-1524.
Savitski, M. M. et al. (2014) ‘Tracking cancer drugs in living cells by thermal profiling of the proteome’, Science 346(6205). doi: 10.1126/science.1255784.
Strønen, E. et al. (2016) ‘Targeting of cancer neoantigens with donor-derived T cell receptor repertoires’, Science 352(6291): 1337-1341. doi: 10.1126/science.aaf2288.
Tummino, P. J. and Copeland, R. A. (2008) ‘Residence Time of Receptor—Ligand Complexes and Its Effect on Biological Function’, Biochemistry 47(20): 5481-92. doi: 1021/bi8002023.
Yewdell, J. W., Reits, E. and Neefjes, J. (2003) ‘Making sense of mass destruction: Quantitating MHC class I antigen presentation’, Nat Rev Immunol, 3(12): 952-961. doi: 10.1038/nri1250.
Maclean B. et al. (2010) ‘Skyline: An Open Source Document Editor for Creating and Analyzing Targeted Proteomics Experiments’, Bioinformatics 26(7): 966-968. doi: 10.1093/bioinformatics/btq054.
Demichev V. et al. (2020) ‘DIA-NN: Neural Networks and Interference Correction Enable Deep Proteome Coverage in High Throughput’, Nat Methods 17(1): 41-44. doi: 10.1038/s41592-019-0638-x.
Rock, K. L., Reits, E, and Neefjes J. (2016), ‘Present Yourself! By MHC Class I and MHC Class II Molecules’, Trends in Immunology, 37(11): 724-737.
Neefjes, J, Jongsma, Paul, P and Bakke, O (2011), ‘Towards a systems understanding of MHC class I and MHC class II antigen presentation’, Nature Reviews Immunology 11(12): 823-836.

1. A method for identification of at least one malignant cell-derived peptide, which comprises or consists of a potential T-cell epitope that binds to at least one MHC molecule in an individual, which harbours the malignant cell, the method comprising
a) comparing proteinaceous expression products of said individual's non-malignant cells with proteinaceous expression products of said individual's malignant cells and identifying a set of proteinaceous expression products that are expression products of the malignant cells but not of the non-malignant cells, and
b) identifying the at least one malignant cell-derived peptide as one having 1) an amino acid sequence, which is present in a proteinaceous expression product in the set and not present in any expression product of the non-malignant cells, and 2) a high likelihood of being a natural product of antigen processing and an effective binder of the at least one MHC molecule when compared to the likelihood of other peptides having amino acid sequences present in a proteinaceous expression product in the set,
wherein likelihood in step b is determined by including evaluation of the stability of binding between the at least one peptide and the at least one MHC molecule.

2. The method according to claim 1, wherein step a) comprises identification of DNA sequences of expressed genes in the genomic DNA from the individual's malignant and non-malignant cells.

3. The method according to claim 1, wherein step a comprises identifying mRNA sequences from the individual's malignant and non-malignant cells.

4. The method according to claim 2, wherein the amino acid sequences of the protein expression products are deduced from the DNA and/or mRNA sequences.

5. (canceled)

6. (canceled)

7. (canceled)

8. (canceled)

9. The method according to claim 1, wherein step b) comprises inputting the sequences of the proteinaceous expression products into a computer or computer system, which
I. generates amino acid sequences of peptides from the sequences of the proteinaceous expression products by a method comprising 1) subjecting the sequences of the proteinaceous expression products to fragmentation in accordance with the sequence specificity of proteolytic enzymes involved in antigen processing, and/or 2) comparing the sequences of the proteinaceous expression products with known amino acid sequences and the known products of antigen processing thereof, and/or
II. is executing code for an artificial neural network, which identifies amino acid sequences of potential T-cell epitopes on the basis of a training set, which comprises amino acid sequences of known protein antigens and their known T-cell epitopes, and optionally MHC restriction.

10. (canceled)

11. (canceled)

12. The method according to claim 1, wherein the high likelihood is among the top 50% of likelihoods determined, such as among the top 60, 70, 80, and 90%.

13. The method according to claim 12, wherein the high likelihood is selected from the top 50 likelihoods, such as the top 40, top 30, and the top 25 likelihoods.

14. The method according to claim 9, wherein step b comprises option II and wherein the training set further comprises a data set comprising:
a plurality of amino acid sequences of peptides that are presented by at least one MHC molecule as natural products of antigen processing of protein,
for each of the plurality of amino acid sequences of peptides, a score for the stability of binding between the peptide and at least one MHC molecule, and, optionally,
a plurality of amino acid sequences from irrelevant peptides that are not presented by the at least one MHC molecule.

15. The method according to claim 14, wherein the score for the stability is
a decay constant for binding between the peptide and the at least one MHC molecule at a selected temperature, or any value being a strictly increasing or decreasing function of the decay constant such as the half-life or the mean lifetime of the peptide binding to the MHC molecule, or
a Tₘ value for binding between the peptide and the at least one MHC molecule for a selected period of time, or any strictly increasing or decreasing function thereof.

16. The method according to claim 14, wherein the score for stability of binding between the peptide and the at least one MHC molecule is determined by mass spectrometry (MS) analysis of peptides eluted from complexes with MHC molecules, which have been subjected to incubation at defined physicochemical conditions, where incubation time varies between the plurality of samples and where the physicochemical conditions are kept constant between the plurality of samples, or incubation at defined physicochemical conditions, where the incubation time is kept constant between the plurality of samples and where the physicochemical conditions vary between the plurality of samples.

17. The method according to claim 1, wherein the score for stability is a probability score indicating the likelihood that the peptide binds stably to the at least one MHC molecule at in vivo physiological conditions.

18. The method according to claim 17, wherein the score for stability of binding between the peptide and the at least one MHC molecule is determined by analysis of mass spectrometry (MS) data from peptides eluted from complexes with MHC molecules, wherein the complexes have been subjected to incubation at defined physicochemical conditions for a period of time.

19. The method according to claim 1, wherein the evaluation of stability of binding between the peptide and the least one MHC molecule is based on a data set defined in claim 14.

20. The method according to claim 19, wherein the data set defined in claim 14 is obtained by a method entailing quantitative determination of stability of binding between at least one peptide and an MHC molecule, comprising the subsequent steps of
a) preparing a plurality of samples of cell lysates comprising complexes between MHC molecules and peptides, where the lysates are obtained from a plurality of MHC expressing cells (preferably human cells) that have naturally processed said peptides from protein antigens,
b) subjecting the plurality of samples to the conditions of
i) incubation at defined physicochemical conditions, where incubation time varies between the plurality of samples and where the physicochemical conditions are kept constant between the plurality of samples, or
ii) incubation at defined physicochemical conditions, where the incubation time is kept constant between the plurality of samples and where the physicochemical conditions vary between the plurality of samples,
c) isolating complexes between MHC molecules and peptides from the plurality of samples,
d) determining, by mass spectrometric analysis, the at least one peptide's relative quantities in the plurality of samples after step c), and
deriving at least one stability score for the at least one peptide based on the quantities determined in step d).

21. The method according to claim 19, wherein the data set defined in claim 17 is obtained by a method entailing determination of stability of binding between at least one peptide and an MHC molecule, comprising the subsequent steps of determination of binding between at least one peptide and an MHC molecule by
I) preparing at least one sample of cell lysates comprising complexes between MHC molecules and peptides, where the lysates are obtained from a plurality of MHC expressing cells (preferably human cells) that have naturally processed said peptides from protein antigens, wherein the at least one sample of cell lysates is prepared at a temperature>4° C. and/or wherein the at least one sample of cell lysates is/are incubated for a period of time after obtaining the cell lysates at defined physicochemical conditions at a temperature>0° C.,
II) determining, by mass spectrometric analysis, whether the at least one peptide is present as part of a complex in the at least one sample after step I).

22. The method according to claim 1, wherein the at least one MHC molecule is an MHC Class I molecule or an MHC Class II molecule.

23. The method according to claim 1, wherein the at least one MHC molecule is an HLA molecule.

24. A method for preparing a personalized immunogenic composition for an individual, such as a human patient, suffering from a malignant neoplastic disease, the method comprising the sequential steps of extraction of genetic material from malignant cells and from normal cells in the patient, wherein the genetic material is genomic DNA and/or mRNA, identification of RNA sequences or DNA sequences of expressed genes in the genomic DNA from the individual's malignant and non-malignant cells, deducing amino acid sequences of the protein expression products from the RNA/DNA sequences, identification of at least one malignant cell-derived peptide according to the method of claim 1, and subsequently
admixing the at least one malignant cell-derived peptide with a pharmaceutically acceptable carrier, diluent, vehicle, and/or excipient, or
preparing a polypeptide, which comprises amino acid sequence(s) of the at least one malignant cell-derived peptide and admixing the polypeptide with a pharmaceutically acceptable carrier, diluent, vehicle, and/or excipient, or
admixing a nucleic acid, such as a plasmid, which comprises nucleotide sequence(s) encoding as expressible product(s) the at least one peptide, with a pharmaceutically acceptable carrier, diluent, vehicle, and/or excipient, or
admixing a nucleic acid, such as a plasmid, comprises a nucleotide sequence which encodes as an expressible product a polypeptide comprising the amino acid sequence(s) of the at least one peptide, with a pharmaceutically acceptable carrier, diluent, vehicle, and/or excipient, or
admixing a microorganism or virus, preferably attenuated and/or non-pathogenic, which is capable of expressing nucleotide sequences encoding the amino acid sequences of the at least one malignant cell-derived peptide, with a pharmaceutically acceptable carrier, diluent, vehicle, and/or excipient, or
admixing a microorganism of virus, preferably attenuated and/or non-pathogenic, which is capable of expressing a nucleotide sequence encoding a polypeptide comprising the amino acid sequences of the at least one malignant cell-derived peptide, with a pharmaceutically acceptable carrier, diluent, vehicle, and/or excipient.

25. (canceled)

26. The method according to claim 24, which also comprises admixing with an immunological adjuvant.

27. A method for therapeutically treating an individual, such as a human patient, suffering from a malignant neoplasm, the method comprising administering an effective amount of a personalized immunogenic composition prepared according to claim 24 to the individual.

28. (canceled)

29. (canceled)

30. The method according to claim 27, which comprises a plurality of administrations, such as in the form of a prime-boost dosage regimen or a burst dosage regimen.

31. The method according to claim 27, wherein the immunogenic composition is administered parenterally, such as via injection, either subcutaneously, intramuscularly, or transdermally/transcutaneously.

32. A computer or computer system comprising
a. an interface for inputting amino acid sequences data and/or nucleotide sequences,
b. if the interface allows input of nucleotide sequences, executable code for identifying coding sequences in nucleotide sequences and generating encoded amino acid sequences therefrom,
c. a storage segment for storing amino acid sequences provided via input from the interface in a and/or the executable code in b or for storing unique identifiers of the amino acid sequences,
d. executable code, which generates amino acid sequences of peptides, the amino acid sequences of which are extracted from the storage segment in c or from source(s) identified by the unique identifiers,
e. executable code for an artificial neural network, which
i. evaluates amino acid sequences of potential T-cell epitopes on the basis of a training set comprising a plurality of amino acid sequences of peptides that are presented by at least one MHC molecule as natural products of antigen processing of protein, and for each of the plurality of amino acid sequences of peptides, a score for the stability of binding between the peptide and the at least one MHC molecule, and
ii. assigns a score of likelihood that an amino acid sequence generated by the executable code in d is an amino acid sequence of a peptide which is a natural product of antigen processing and a strong binder of the at least one MHC molecule, and
f. a storage segment for storing and/or an interface for output of the scores of likelihood generated by the artificial neural network in e, so as to enable comparison between the amino acid sequences generated by the executable code in d with respect to their scores of likelihood.

33. The computer or computer system according to claim 31, wherein the interface in a) is selected from a manual input device, such as a keyboard, a voice recognition system, a reader of information on a storage medium, a database connection, and a data acquisition system.

34. The computer or computer system according to claim 31 wherein the training set comprises amino acid sequences of peptides that are presented by MHC Class I molecules.

35. The computer system according to claim 31, which further comprises executable code and storage necessary for carrying out the method of claim 1.

36. A computer-readable, preferably non-transitory, medium storing computer-executable code for identifying potential T-cell epitopes, wherein the code is executable by a computer processor to identify RNA sequences or DNA sequences of expressed genes in genomic DNA from malignant and non-malignant cells, deducing amino acid sequences of the protein expression products from the RNA/DNA sequences, comparing proteinaceous expression products non-malignant cells with proteinaceous expression products of malignant cells and identifying a set of proteinaceous expression products that are expression products of the malignant cells but not of the non-malignant cells, and identifying the at least one malignant cell-derived peptide as one having 1) an amino acid sequence, which is present in a proteinaceous expression product in the set and not present in any expression product of the non-malignant cells, and 2) a high likelihood of being a natural product of antigen processing and an effective binder of the at least one MHC molecule when compared to the likelihood of other peptides having amino acid sequences present in a proteinaceous expression in the set, wherein likelihood in step b is determined by including evaluation of the stability of binding between the at least one peptide and the at least one MHC molecule.

37. The computer readable medium according to claim 36, wherein the executable code further
I. generates amino acid sequences of peptides from the sequences of the proteinaceous expression products by 1) subjecting the sequences of the proteinaceous expression products to fragmentation in accordance with the sequence specificity of proteolytic enzymes involved in antigen processing, and/or by 2) comparing the sequences of the proteinaceous expression products with known amino acid sequences and known products of antigen processing thereof, and/or
II. comprises code for an artificial neural network, which identifies amino acid sequences of potential T-cell epitopes on the basis of a training set, which comprises amino acid sequences of known protein antigens and their known T-cell epitopes.

38. The computer readable medium according to claim 36, wherein the executable code further implements the method steps defined in claim 1.


SEQUENCE LISTING (42 sequences)

SEQ ID NO: 1 - protein, 10 aa, Homo sapiens
TLIDLGITKV

SEQ ID NO: 2 - protein, 10 aa, Homo sapiens
FLLGPRLVLA

SEQ ID NO: 3 - protein, 9 aa, Homo sapiens
LLLPGPSAA

SEQ ID NO: 4 - protein, 9 aa, Homo sapiens
SLIGHLQTL

SEQ ID NO: 5 - protein, 9 aa, Homo sapiens
KLLEPVLLL

SEQ ID NO: 6 - protein, 9 aa, Homo sapiens
IMDDKSLNI

SEQ ID NO: 7 - protein, 9 aa, Homo sapiens
YLPEDFIRV

SEQ ID NO: 8 - protein, 9 aa, Homo sapiens
TLYPGRVIL

SEQ ID NO: 9 - protein, 9 aa, Homo sapiens
SVDSKPPVI

SEQ ID NO: 10 - protein, 9 aa, Homo sapiens
LLFPESIRV

SEQ ID NO: 11 - protein, 9 aa, Homo sapiens
TLFPVRLLV

SEQ ID NO: 12 - protein, 11 aa, Homo sapiens
GLTSTDLLFHL

SEQ ID NO: 13 - protein, 9 aa, Homo sapiens
SPSLPSRTL

SEQ ID NO: 14 - protein, 9 aa, Homo sapiens
APSRNGMVL

SEQ ID NO: 15 - protein, 10 aa, Homo sapiens
APFQGDQRSL

SEQ ID NO: 16 - protein, 9 aa, Homo sapiens
VPRNYDPAL

SEQ ID NO: 17 - protein, 10 aa, Homo sapiens
APIGSRGPQL

SEQ ID NO: 18 - protein, 9 aa, Homo sapiens
IPKSAVGEL

SEQ ID NO: 19 - protein, 9 aa, Homo sapiens
RPAVIGDVL

SEQ ID NO: 20 - protein, 10 aa, Homo sapiens
HPIEGSTTVF

SEQ ID NO: 21 - protein, 9 aa, Homo sapiens
GPAPVGRFF

SEQ ID NO: 22 - protein, 10 aa, Homo sapiens
SPDTAIRSGL

SEQ ID NO: 23 - protein, 9 aa, Homo sapiens
APHDYGLIL

SEQ ID NO: 24 - protein, 9 aa, Homo sapiens
IPNWARQDL

SEQ ID NO: 25 - protein, 9 aa, Homo sapiens
HWDPQEVTL

SEQ ID NO: 26 - protein, 9 aa, Homo sapiens
SFDETVTHF

SEQ ID NO: 27 - protein, 9 aa, Homo sapiens
HFDDFVTNL

SEQ ID NO: 28 - protein, 9 aa, Homo sapiens
TFDDIVHSF

SEQ ID NO: 29 - protein, 9 aa, Homo sapiens
SFEEKPVYI

SEQ ID NO: 30 - protein, 9 aa, Homo sapiens
FTDEESRVF

SEQ ID NO: 31 - protein, 8 aa, Homo sapiens
SFDPSRTF

SEQ ID NO: 32 - protein, 9 aa, Homo sapiens
AHDVDPIEL

SEQ ID NO: 33 - protein, 9 aa, Homo sapiens
LYDPVISKL

SEQ ID NO: 34 - protein, 8 aa, Homo sapiens
FNDTFVHV

SEQ ID NO: 35 - protein, 9 aa, Homo sapiens
VTDPRVVDL

SEQ ID NO: 36 - protein, 9 aa, Homo sapiens
LFDDIDHNM

SEQ ID NO: 37 - protein, 9 aa, Homo sapiens
RLFDEPQLA

SEQ ID NO: 38 - protein, 9 aa, Homo sapiens
SLLESVQKL

SEQ ID NO: 39 - protein, 9 aa, Homo sapiens
FLFQEPRSI

SEQ ID NO: 40 - protein, 11 aa, Homo sapiens
ILLPEPSIRSV

SEQ ID NO: 41 - protein, 10 aa, Homo sapiens
TLITDGMRSV

SEQ ID NO: 42 - protein, 9 aa, Homo sapiens
FLDENVHFF